(19) United States
(12) Patent Application Publication
(10) Pub. No.: US 2024/0002534 A1
(43) Pub. Date: Jan. 4, 2024

(54) ARGINASE 1 BINDERS FOR INHIBITING ARGINASE 1 ACTIVITY

(71) Applicants: Marc Andre BAILLY, South San Francisco, CA (US); Kalyan CHAKRAVARTHY, Woburn, MA (US); Ghassan Najib FAYAD, South San Francisco, CA (US); Laurence FAYAT-DILMAN, South San Francisco, CA (US); Esther KOFMAN, South San Francisco, CA (US); Masahisa HANDA, South San Francisco, CA (US); Jennifer E. O'NEIL, Dedham, MA (US); Rachel L. PALTE, Boston, MA (US); Giovanna SCAPIN, New York, NY (US); Shahriar Shane TAREMI, Boston, MA (US); MERCK SHARP & DOHME LLC, Rahway, NJ (US)

(72) Inventors: Marc Andre Bailly, San Mateo, CA (US); Kalyan Chakravarthy, Woburn, MA (US); Ghassan Najib Fayad, Waban, MA (US); Laurence Fayadat-Dilman, San Carlos, CA (US); Esther Kofman, San Jose, CA (US); Masahisa Handa, Berkeley, CA (US); Jennifer E. O'Neil, Dedham, MA (US); Rachel Lynn Palte, Melrose, MA (US); Giovanna Scapin, New York, NY (US); Shahriar Shane Taremi, Cambridge, MA (US)

(73) Assignee: MERCK SHARP & DOHME LLC, Rahway, NJ (US)

(21) Appl. No.: 18/253,702

(22) PCT Filed: Nov. 22, 2021
(86) PCT No.: PCT/US2021/060240

§ 371 Date: May 19, 2023

Related U.S. Application Data
(60) Provisional application No. 63/119,265, filed on Nov. 30, 2020.

(51) Int. Cl. C07K 16/40; C07K 16/28; C12N 15/85

(52) U.S. Cl. CPC C07K 16/40; C07K 16/2818; C07K 16/2827; C12N 15/85; C12N 2800/107

(57) ABSTRACT

Arginase 1 binders that inhibit the activity of human Arginase 1 (hArg1) and comprise human antibodies and antigen-binding fragments thereof comprising human VH and VL are described. These Arginase 1 binders present an alternative mechanism for inhibiting hArg1 activity and highlight the ability to utilize binders as probes in the discovery and development of peptide and small molecule inhibitors for enzymes in general.

### BACKGROUND OF THE INVENTION

### (1) Field of the Invention

The present invention relates to Arginase 1 binders that inhibit activity of human arginase 1 (hArg1), which comprise human anti-hArg1 antibodies and antigen-binding fragments thereof.

### (2) Description of Related Art

Human Arginase 1 is a metalloenzyme that catalyzes the hydrolysis of L-arginine to L-ornithine and urea and is a critical endogenous regulator of the immune system and a key player in T-cell function. This enzyme is constitutively expressed by myeloid derived suppressor cells (MDSCs), which are known immune system regulators. MDSCs have emerged as a key mediator of immunosuppression in human T-cells biology leading to significant decreases in the induction of antitumor activity (Bronte et al., J. Immunother. 24, 431-446 (2001); Kusmartsev & Gabrilovich, Cancer Immunol. Immunother. 51, 293-298 (2002); Serafini et al., Semin. Cancer Biol. 16, 53-65 (2006); Kumar et al., Trends Immunol. 37, 208-220 (2016)). hArg1 catalyzes the degradation of the conditionally essential amino acid L-arginine into L-ornithine and urea in the final step of the urea cycle (Kumar et al., Ibid; Bronte et al., Trends Immunol. 24, 301-305 (2003); Rodriguez et al., Cancer Res. 64, 5839-5849 (2004)). This enzyme is present both intracellularly and excreted into the extracellular environment in a paracrine manner, with extracellular hArg1 maintaining its ability to deplete L-arginine (Pudlo et al., Med. Res. Rev. 37, 475-513 (2017); Sahin et al., J. Immunol. 193, 1717-1727 (2014); Munder, Br. J. Pharmacol. 158, 638-651 (2009); Wu et al., Amino Acids 37, 153-168 (20089)). As T-cells are dependent on L-arginine for growth and proliferation, its depletion leads to the effective suppression of T-cell immune responses and consequently supports the proliferation of tumor cells both in vitro and in vivo (Kumar et al. op. cit.; Rodriquez et al., op. cit. Activation of lymphocytes, specifically T-cells, via therapeutics targeted at immune checkpoint molecules enhances tumor cell killing and has led to long-lasting responses across various cancers (Wei et al., Cancer Discov. 8, 1069-1086 (2018)). High levels of hArg1 activity have been correlated with various types of cancer (Rodriquez et al., op. cit .; Kusmartsev & Gabrilovich, Cancer Immunol Immunother. 55, 237-245 (2006)).

hArg1 is a trimeric metalloenzyme in which each monomer is approximately 35 kDa in size with an extended, narrow active site approximately 15 Å deep that is terminated by two catalytic manganese (Mn) ions 3.3 Å apart (Ash, J. Nutr. 134, 2760-2764 (2004)). Several residues within the active site are critical for bridging the two Mn ions and in binding L-arginine (Ash, op. cit.: Costanzo et al., Proc. Natl. Acad. Sci. U.S.A. 102, 13058-13063 (2005) and have been the main target of small molecule inhibitors (Ilies et al., J. Med. Chem. 54, 5432-5443 (2011); Cox et al., Nat. Struct. Biol. 6, 1043-1047 (1999); Van Zandt & Jagdmann Jr., Ring Constrained Analogs As Arginase Inhibitors. 1-21 (2015); Van Zandt et al .. J. Med. Chem. 62, 8164-8177 (2019); Van Zandt et al., J. Med. Chem. 56, 2568-2580 (2013); Mitcheltree et al., ACS Med. Chem. Lett. 11, 582-588 (2020); Steggerda et al., Cancer 5, 1-18 (2017)). Efforts to discover pharmacological agents to inhibit hArg1 have been focused on amino acid-derived small molecules of usually less than 350 Da (Pudlo, op. cit.) that are able to enter and bind to residues within the hArg1 (Costanzo, op. cit.) active site. One avenue not previously described in literature for hArg1 inhibition is the use of therapeutic antibodies.

Monoclonal antibodies (mAbs) both in monotherapy and in combination regiments has emerged as one of the fastest growing and most effective therapeutic strategies for the treatment of solid tumors and hematological diseases. Between 2015 and 2017, the U.S. Food and Drug Administration approved 27 therapeutic mAbs (Tsumoto et al., Immunotherapy 11, 119-127 (2019)) increasing the total of clinically used mAbs and biosimilars in 2017 to 57 and 11, respectively (Grilo & Mantalaris, Trends Biotechnol. 37, 9-16 (2019)). As of late 2019, numerous companies were supporting over 550 novel antibody therapeutics in early phase clinical trials, with approximately half of these against oncology targets (Kaplon et al., MAbs 12, 1-24 (2020)).

Numerous published studies focused on the use of antibody fragments such as nanobodies, antigen-binding fragments (Fabs), and single-chain variable domain fragments (scFvs) as potent inhibitors of enzyme activity 25-34 (Dahms et al., Sci. Rep. 6, 1-7 (2016); Ganesan et al., Structural and mechanistic insight into how antibodies inhibit serine proteases. Biochem. J. 430, 179-189 (2010); Cinader. & Lafferty, Immunology 7, 342-362 (1964); Lauwereys et al., EMBO J. 17, 3512-3520 (1998); Holliger & Hudson, Nat. Biotechnol. 23, 1126-1136 (2005); Remy et al., Eur. J. Biochem. 231, 651-658 (1995); Oyen et al., J. Mol. Biol. 407, 138-148 (2011); Ganesan, Structure 17, 1614-1624 (2009); De Genst et al., Proc. Natl. Acad. Sci. U.S.A. 103, 4586-4591 (2006); Koschubs, T. et al. Biochem. J. 442, 483-494 (2012)). The proposed mechanisms of inhibition by antibodies include adaptation to the catalytic site; adaptation to a site other than but near to the catalytic center thereby causing steric hindrance; aggregation of the antigen-antibody complex leading to steric hindrance by the structure of the aggregate; and interference with multimerization that may inhibit enzyme activity (Cinader, Annu. Rev. Microbiol. 11, 371-390 (1957)). Nevertheless, as noted by others, despite the myriad antibodies that have been and could be developed, the number of full-length monoclonal antibodies acting as enzyme inhibitors is “disappointingly low.” (Lauwereys, op. cit.). MAbs excel in their ability to bind an antigen with high specificity and potency and function mainly by binding to large, flat surfaces on some receptors and protein:protein interaction surfaces that traditional small molecules cannot bind with suitable potency. Full-length neutralizing antibodies often lack the ability to access the narrow clefts and active site pockets of traditional enzymes due to their larger size, which often eliminates the ability to inhibit enzymatic activity.

Arginase-targeted therapies have been pursued across several disease areas including immunology, oncology, nervous system dysfunction, and cardiovascular dysfunction and diseases. Currently, all published hArg1 inhibitors are small molecules usually less than 350 Da in size.

### BRIEF SUMMARY OF THE INVENTION

The present invention provides Arginase 1 binders that inhibit human arginase 1 (hArg1) comprising human anti-hArg1 antibodies and antigen-binding fragments thereof obtained from chimeric antibodies comprising human heavy chain variable domain (VH) and light chain variable domain (VL) on mouse IgG and kappa constant domains, respectively, that were produced by transgenic mice that had been exposed to hArg1. The Arginase 1 binders inhibit hArg1 through orthosteric and allosteric mechanisms. These Arginase 1 binders may be useful for treating cancers and proliferative diseases.

The present invention further provides an Arginase 1 binder comprising (a) three complementarity determining regions (CDRs) of an antibody VH comprising the amino acid sequence set forth in SEQ ID NO: 2; and the three CDRs of an antibody VL comprising the amino acid sequence set forth in SEQ ID NO: 3. CDR sequences can be determined using a Kabat, Chothia, Kabat+Chothia, AbM, ImMunoGeneTics (IMGT), or Contact numbering scheme.

In a further embodiment, the Arginase 1 binder specifically binds to an arginase 1 trimer to form a complex comprising three Arginase 1 binders and one arginase trimer, and inhibits arginase 1 activity.

In a further embodiment of the Arginase 1 binder, (a) the VH CDRs comprise a VH-CDR1 comprising the amino acid sequence set forth in SEQ ID NO: 4, a VH-CDR2 comprising the amino acid sequence set forth in SEQ ID NO: 5, and a VH-CDR3 comprising the amino acid sequence set forth in SEQ ID NO: 6; and (b) the VL CDRs comprise a VL-CDR1 comprising the amino acid sequence set forth in SEQ ID NO: 17, a VL-CDR2 comprising the amino acid sequence set forth in SEQ ID NO: 18, and a VL-CDR3 comprising the amino acid sequence set forth in SEQ ID NO: 19, wherein the CDR sequences are defined by Kabat.

In a further embodiment, the Arginase 1 binder comprises a VH comprising the amino acid sequence set forth in SEQ ID NO: 2 and a VL comprising the amino acid sequence set forth in SEQ ID NO: 3.

In a further embodiment, the Arginase 1 binder comprises a heavy chain constant domain of the IgG1, IgG2, IgG3, or IgG4 isotype. In a further embodiment of the Arginase 1 binder, the antibody comprises a heavy chain constant domain of the IgG1, IgG2, IgG3, or IgG4 isotype wherein the Fc domain comprises one or more mutations that render the Fc domain effector silent.

In a further embodiment, the light chain may comprise a human kappa light chain constant domain or a lambda light chain constant domain. In further embodiments disclosed herein, the light chain may comprise a human kappa light chain constant domain comprising SEQ ID NO: 58 or a lambda light chain constant domain comprising SEQ ID NO: 64.

The present invention provides an Arginase 1 binder that is an antibody or an antigen binding fragment thereof comprising two identical Fabs, a first Fab comprising a first heavy chain variable domain (VH) and a first light chain variable domain (VL) and a second Fab comprising a second VH and a second VL, wherein the Arginase 1 binder binds to an arginase 1 trimer comprising three arginase 1 monomers, and wherein the first Fab VH and VL bind to an epitope of a monomer of the arginase 1 trimer and the second Fab VH and VL do not detectably bind an epitope of a monomer of an arginase 1 trimer. The 1:3 ratio of one trimer to three antibodies may be identified using both isothermal titration calorimetry (ITC) and size exclusion chromatography with multi-angle light scattering (SECMALS) and/or cryo-electron microscopy.

In a further embodiment of the Arginase 1 binder, (a) the VH of the first Fab binds to amino acids Arg21, Ser281, Gly22, Ser281, Leu282, Glu26, Pro59, Lys17, Asp57, Ile58, Pro59, Pro20, Lys68, Ser16, Gln19, Glu25, Asn69, and Glu25 of a monomer of the arginase 1 trimer: and (b) the VL of the first Fab binds to amino acids Pro54, Phe55, Asp57, Pro59, and Asn60 of a monomer of the arginase 1 trimer.

The present invention further provides a composition comprising an Arginase 1 binder disclosed herein and a pharmaceutically acceptable carrier.

The present invention further provides a method for treating cancer or proliferative disease in an individual in need thereof comprising administering to the individual a therapeutically effective amount an Arginase 1 binder disclosed herein or a composition disclosed herein to treat the cancer or a proliferative disease.

The present invention further provides an arginase 1 binder or composition disclosed herein for treatment of cancers or proliferative diseases.

The present invention further provides for the use of an Arginase 1 binder disclosed herein for the manufacture of a medicament for treating cancer or proliferative disease.

The present invention further provides a combination therapy for treating a cancer or proliferative disease comprising an arginase 1 binder or composition disclosed herein and a therapeutic agent. In a further embodiment, the therapeutic agent is a chemotherapy agent or a therapeutic antibody. In a further still embodiment, the antibody is an anti-PD1 or anti-PD-L1 antibody.

The present invention further provides a nucleic acid molecule encoding the VH of an Arginase 1 binder disclosed herein and/or VL of an Arginase 1 binder disclosed herein. Further provided is an expression vector comprising one or more of the nucleic acid molecules disclosed herein. Further provided is host cell comprising an expression vector comprising one or more of the nucleic acid molecules disclosed herein.

The present invention further provides a method for producing an Arginase 1 binder comprising (a) providing a host cell comprising an expression vector comprising one or more of the nucleic acid molecules disclosed herein; (b) cultivating the host cell in a medium under conditions suitable for expressing the Arginase 1 binder; and (c) isolating the Arginase 1 binder from the medium.

In any one of the embodiments disclosed herein, the Arginase 1 binder further comprises a heavy chain constant domain of the IgG1, IgG2, IgG3, or IgG4 isotype. In a further embodiment thereof, the heavy chain constant domain of the IgG1, IgG2, IgG3, or IgG4 isotype comprises an Fc domain comprising one or more mutations that render the Fc domain effector-silent.

In any one of the embodiments disclosed herein, the light chain may comprise a human kappa light chain constant domain or a lambda light chain constant domain. In any one of the embodiments disclosed herein, the light chain may comprise a human kappa light chain constant domain comprising SEQ ID NO: 58 or a lambda light chain constant domain comprising SEQ ID NO: 64.

### BRIEF DESCRIPTION OF THE DRAWINGS

FIG. 1A. Shows the CDRs of the VH comprising the amino acid sequence set forth in SEQ ID NO: 2 as defined according to Kabat, Chothia, and Kabat+Chothia.

FIG. 1B. Shows the CDRs of the VH comprising the amino acid sequence set forth in SEQ ID NO: 2 as defined according to AbM, IMGT, and Contact.

FIG. 1C. Shows the CDRs of the VL comprising the amino acid sequence set forth in SEQ ID NO: 3 as defined according to Kabat, Chothia, Kabat+Chothia, AbM, IMGT, and Contact.

FIG. 2. Shows the potency and multiplicity of infection (MOI) for mAb5.

FIG. 3. Shows a dose response curve as determined by TOGA for mAb5.

FIG. 4. Shows the distance (Å) over 100 ns of molecular dynamics simulations between hArg1's Arg21 (guanidino C atom CZ) and mAb5's Asp30 (carboxylate C atom GC).

FIG. 5A. Overall complex formation and epitope mapping for mAb5 1:3 complex. Left panel: density for the bottom half of the complex is almost completely absent. In masked maps at very low contour it is possible to visualize some density for the other Fabs and the Fcs, but the Fabs on the bottom half appear to be in a different conformation and closer to each other with no density for hArg1 trimer present. Middle panel: shows an overview of how the 1:3 hArg1 to mAb5 complex assembles. The three monomers of the hArg1 trimer are shown as monomers A, B, and C, and the mAb5 heavy chain and light chain are colored dark grey and light grey, respectively. The protein surfaces are shown for the top half of the complex, and the bottom half Fabs and one Fc shown by the ovals. Right panel: A depiction of the complex for ease of interpretation. Each antibody interacts with only one hArg1 monomer and these interactions are symmetric around the trimer.

FIG. 5B. Left panel: details of the interactions between the Fab and the hArg1 monomer are shown, with several residues involved in hydrogen bonding interactions labeled. Tyr91 is present in LC CDR3, Arg101 and Tyr105 are present in HC-CDR3, and Arg21 of the hArg1 monomer are shown (Arg101 and Tyr105 are Arg97 and Tyr100a according to Kabat numbering of VH). Right panel: surface view of one of the hArg1 monomers shows that the active site is fully exposed in this complex. The binuclear active site manganese ions (Mn) are shown as spheres.

FIG. 6. Comparison of an hArg1 loop containing amino acid residues Lys16-Val24 when bound to mAb5 and small molecule inhibitor (SMI). Left panel: an overlay of the hArg1 loop showing its position when hArg1 is bound to mAb5 or SMI. When bound to mAb5, the positioning of the loop is altered compared to when hArg1 is bound to an SMI. Middle panel: a closeup of Arg21 position highlights the difference in orientation of Arg21 when hArg1 is bound to mAb5 compared to when the hArg1 is bound by an SMI: the Arg21 moves outward by 5.8-6.4 Å when hArg1 is bound by mAb5. Right panel: the Arg21 interacts with the Asp30 of the mAb5 heavy chain which sits approximately 2.2-3.5 Å away.

FIG. 7. Size and shape comparison of an hArg1:mAb5trimer 1:3 complex. Left Panel: the mAb5 complex shows two separate measurements: one from the Arg222 C-α of one hArg1 monomer to the terminal Cys214 residue of the mAb5 LC (110 Å) and the other from the Arg222 C-α of the hArg1 monomer to the terminal Asp224 residue of the mAb5 HC (115 Å). The three monomers of the hArg1 trimer are shown as monomers A, B, and C, and the mAb5 heavy chain and light chain are colored dark grey and light grey, respectively. Shown are the Fab1 and Fab2 of each of three mAb4 antibodies (Fab, Fab′, and Fab″). The Fc and the Fab2 of the antibodies are not shown. Right panel: the binding of Fab1 of the mAb5 to hArg1 monomer A is depicted in cartoon form and illustrate how Fab1 binds to monomer A.

FIG. 8. Predominant N-linked glycans for monoclonal antibodies produced in Chinese hamster ovary cells (CHO N-linked glycans) and in engineered yeast cells (engineered yeast N-linked glycans): squares: N-acetylglucosamine (GlcNac); circles: mannose (Man); diamonds: galactose (Gal); triangles: fucose (Fuc).

FIG. 9. Shows the amino acid sequence for human Arginase 1 (SEQ ID NO. 1). The amino acids boxed are amino acids at the mouth of the active site and the amino acids underlined are those amino acids involved in monomer:monomer interactions.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

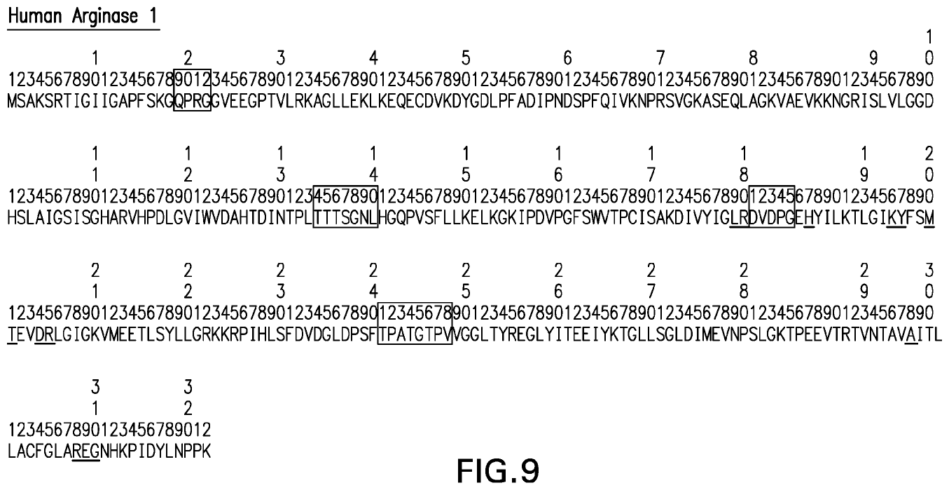

As used herein, the term “Arginase” refers to a manganese-containing enzyme (EC 3.5.3.1, arginine amidinase, canavanase, L-arginase, arginine transamidinase) that catalyzes the conversion of arginine to ornithine+urea. It is the final enzyme of the urea cycle and is ubiquitous to all domains of life. Two isozymes of this arginase exist: arginase 1, which functions in the urea cycle, and is located primarily in the cytoplasm of hepatocytes (liver cells); and. arginase 2, which may be involved in the regulation of intracellular arginine/ornithine levels, and is located in mitochondria of several tissues in the body, with most abundance in the kidney and prostate and at lower levels in macrophages, lactating mammary glands, and brain. The amino acid sequence of human Arginase 1 is set forth in SEQ ID NO: 1 and shown in FIG. 9.

As used herein, the term “hArg1” refers to human arginase 1.

As used herein, the term “Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including KinExA and Biacore. Specific illustrative and exemplary embodiments for measuring binding affinity are described in the following.

As used herein, the term “administration” and “treatment,” as it applies to an animal, human, experimental subject, cell, tissue, organ, or biological fluid, refers to contact of an exogenous pharmaceutical, therapeutic, diagnostic agent, or composition comprising an Arginase 1 binder as disclosed herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subject” refers to a human.

As used herein, the term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “antibody” or “immunoglobulin” as used herein refers to a glycoprotein comprising either (a) at least two heavy chains (HCs) and two light chains (LCs) inter-connected by disulfide bonds, or (b) in the case of a species of camelid antibody, at least two heavy chains (HCs) inter-connected by disulfide bonds. Each HC is comprised of a heavy chain variable region or domain (VH) and a heavy chain constant region or domain. Each light chain is comprised of an LC variable region or domain (VL) and a LC constant domain. In certain naturally occurring IgG, IgD and IgA antibodies, the heavy chain constant region is comprised of three domains, CH1, CH2 and CH3. In general, the basic antibody structural unit for antibodies is a Y-shaped tetramer comprising two HC/LC pairs (2H+2L), except for the species of camelid antibodies comprising only two HCs (2H), in which case the structural unit is a homodimer. Each tetramer includes two identical pairs of polypeptide chains, each pair having one LC (about 25 kDa) and HC chain (about 50-70 kDa) (H+L). Each HC:LC pair comprises one VH: one VL pair. The one VH:one VL pair may be referred to by the term “Fab”. Thus, each antibody tetramer comprises two Fabs, one per each arm of the Y-shaped antibody.

The LC constant domain is comprised of one domain, CL. The human VH includes seven family members: VH1, VH2, VH3, VH4, VH5, VH6, and VH7; and the human VL includes 16 family members: Vκ1, Vκ2, Vκ3, Vκ4, Vκ5, Vκ6, Vλ1, Vλ2, Vλ3, Vλ4, Vλ5, Vλ6, Vλ7, Vλ8, Vλ9, and Vλ10. Each of these family members can be further divided into particular subtypes. The VH and VL can be further subdivided into regions of hypervariability, termed complementarity determining region (CDR) areas, interspersed with regions that are more conserved, termed framework regions (FR). Each VH and VL is composed of three CDR regions and four FR regions, arranged from amino-terminus to carboxy-terminus in the following order: FR1, CDR1, FR2, CDR2, FR3, CDR3, FR4. Numbering of the amino acids in a VH or VHH may be determined using the Kabat numbering scheme. See Béranger, et al., Ed. Ginetoux, Correspondence between the IMGT unique numbering for C-DOMAIN, the IMGT exon numbering, the Eu and Kabat numberings: Human IGHG, Created: 17 May 2001, Version: 8 Jun. 2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Béranger, et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al, ILAR Journal 37: 132-141 (1995);
Contact numbering scheme is based on an analysis of the available complex crystal structures (See www.bioinf.org.uk: Prof. Andrew C.R. Martin's Group: Abhinandan & Martin, Mol. Immunol. 45:3832-3839 (2008)).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof. Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur: however, the Cys residues are the most conserved feature.

TABLE 1

| Loop | Kabat | AbM | Chothia¹ | Contact² | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L51 |
| L3 | L89-L97 | L89-L97 | L89-L97 | L89-L96 | L89-L97 |
| H1 | H31-H35B | H26- | H26- | H30-H35B | H26- |
| | (Kabat | H35B | H32 . . . 34 | | H35B |
| | Numbering)³ | | | | |
| H1 | H31-H35 | H26-H35 | H26-H32 | H30-H35 | H26-H33 |
| | (Chothia | | | | |
| | Numbering) | | | | |
| H2 | H50-H65 | H50-H58 | H52-H56 | H47-H58 | H51-H56 |
| H3 | H95-H102 | H95- | H95-H102 | H93-H101 | H93- |
| | | H102 | | | H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire nucleotide sequence of the heavy chain and light chain variable regions are commonly numbered according to Kabat while the three CDRs within the variable region may be defined according to any one of the aforementioned numbering schemes.

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

As used herein, the term “Fc domain”, or “Fc” as used herein is the crystallizable fragment domain or region obtained from an antibody that comprises the CH2 and CH3 domains of an antibody. In an antibody, the two Fc domains are held together by two or more disulfide bonds and by hydrophobic interactions of the CH3 domains. The Fc domain may be obtained by digesting an antibody with the protease papain. Typically, amino acids in the Fc domain are numbered according to the Eu numbering convention (See Edelmann et al., Biochem. 63: 78-85 (1969)).

As used herein, the term “antigen” as used herein refers to any foreign substance which induces an immune response in the body.

As used here, the term “Arginase 1 binder” refers to a polypeptide or protein that binds to arginase 1. An Arginase 1 binder includes but is not limited to a bivalent antibody tetramer (2H+2L), a monovalent antibody (H+L), a bi-specific antibody that targets arginase 1 and another target, a Fab fragment, a Fab′ fragment, a F(ab′)2 fragment, an Fv region, and an ScFv. Unless otherwise indicated, the Arginase 1 binders herein bind to and inhibit the activity of hARG1.

As used herein, the term “Fab fragment” refers to an antigen binder comprising one antibody light chain and the CHI and VH of one antibody heavy chain. The heavy chain of a Fab molecule cannot form a disulfide bond with another heavy chain molecule. A “Fab fragment” can be the product of papain cleavage of an antibody.

As used herein, the term “Fab′ fragment” refers to an antigen binder comprising one antibody light chain and a portion or fragment of one antibody heavy chain that contains the VH and the CHI domain up to a region between the CH1 and CH2 domains, such that an interchain disulfide bond can be formed between the two heavy chains of two Fab′ fragments to form a F(ab′)2 molecule.

As used herein, the term “F(ab′)2 fragment” refers to an antigen binder comprising two antibody light chains and two heavy chains containing the VH and the CH1 domain up to a region between the CH1 and CH2 domains, such that an interchain disulfide bond is formed between the two heavy chains. An F(ab′)2 fragment thus is composed of two Fab′ fragments that are held together by a disulfide bond between the two heavy chains. An “F(ab′)2 fragment” can be the product of pepsin cleavage of an antibody.

As used herein, the term “Fv region” refers to an antigen binder comprising the variable regions from both the heavy and light chains of an antibody, but lacks the constant regions.

As used herein, the term “ScFv” or “single-chain variable fragment” refers to a fusion protein comprising a VH and VL fused or linked together by a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

As used herein, the term “diabody” refers to an antigen binder comprising a small antibody fragment with two antigen-binding regions, which fragments comprise a heavy chain variable domain (VH) connected to a light chain variable domain (VL) in the same polypeptide chain (VH-VL or VL-VH). By using a linker that is too short to allow pairing between the two domains on the same chain, the domains are forced to pair with the complementarity domains of another chain and create two antigen-binding regions. Diabodies are described more fully in, e.g., EP 404,097: WO 93/11161: and Holliger et al. (1993) Proc. Natl. Acad. Sci. USA 90: 6444-6448. For a review of engineered antibody variants generally see Holliger and Hudson (2005) Nat. Biotechnol. 23:1126-1136.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, the term “isolated” antibodies or antigen-binding fragments thereof (e.g., antigen binders such as Arginase 1 binders) are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see. e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, the term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. Genes include both naturally occurring nucleotide sequences encoding a molecule of interest and synthetically derived nucleotide sequences encoding a molecule of interest, for example, complementary DNA (cDNA) obtained from a messenger RNA (mRNA) nucleotide sequence.

As used herein, the term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

As used herein, the term “library” as used herein is, typically, a collection of related but diverse polynucleotides that are, in general, in a common vector backbone. For example, a light chain or heavy chain immunoglobulin library may contain polynucleotides, in a common vector backbone, that encode light and/or heavy chain immunoglobulins, which are diverse but related in their nucleotide sequence: for example, which immunoglobulins are functionally diverse in their abilities to form complexes with other immunoglobulins, e.g., in an antibody display system of the present invention, and bind a particular antigen.

As used herein, the term “polynucleotides” discussed herein form part of the present invention. A “polynucleotide”, “nucleic acid ” or “nucleic acid molecule” include DNA and RNA, single- or double-stranded. Polynucleotides e.g., encoding an immunoglobulin chain or component of the antibody display system of the present invention (e.g., a bait), may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Polynucleotides e.g., encoding an immunoglobulin chain or component of the antibody display system of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist. et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the β-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980)) 242:74-94; and promoter elements from yeast or other fungi such as the Gal 4 promoter, the ADC (alcohol dehydrogenase) promoter. PGK (phosphoglycerol kinase) promoter or the alkaline phosphatase promoter.

As used herein, the terms “vector”, “cloning vector” and “expression vector” include a vehicle (e.g., a plasmid) by which a DNA or RNA sequence can be introduced into a host cell, so as to transform the host and, optionally, promote expression and/or replication of the introduced sequence. Polynucleotides encoding an immunoglobulin chain or component of the antibody display system of the present invention (e.g., a bait) may, in an embodiment of the invention, be in a vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, the term “control sequences” or “regulatory sequences” refers to DNA sequences necessary for the expression of an operably linked coding sequence in a particular host organism. The control sequences that are suitable for expression in eukaryotes, for example, include a promoter, operator or enhancer sequences, transcription termination sequences, and polyadenylation sequences for expression of a messenger RNA encoding a protein and a ribosome binding site for facilitating translation of the messenger RNA.

As used herein, a nucleic acid is “operably linked” when it is placed into a functional relationship with another nucleic acid sequence, e.g., a regulatory sequence. For example, DNA for a presequence or secretory leader is operably linked to DNA for a polypeptide if it is expressed as a preprotein that participates in the secretion of the polypeptide: a promoter or enhancer is operably linked to a coding sequence if it affects the transcription of the sequence: or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., IRNA, RNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, “treat” or “treating” means to administer a therapeutic agent, such as a composition containing any of the Arginase binders of the present invention, topically, subcutaneously, intramuscular, intradermally, or systemically to an individual in need. The amount of a therapeutic agent that is effective to alleviate cancer or proliferative disease in the individual may vary according to factors such as the injury or disease state, age, and/or weight of the individual, and the ability of the therapeutic agent to elicit a desired response in the individual. Whether the therapeutic objective has been achieved can be assessed by the individual and/or any clinical measurement typically used by physicians or other skilled healthcare providers to assess the severity or progression status of the treatment. Thus, the terms denote that a beneficial result has been or will be conferred on a human or animal individual in need.

As used herein, “treatment,” as it applies to a human or veterinary individual, refers to therapeutic treatment, as well as diagnostic applications. “Treatment” as it applies to a human or veterinary individual, encompasses contact of the antibodies or antigen binding fragments of the present invention to a human or animal subject.

As used herein, “therapeutically effective amount” refers to a quantity of a specific substance sufficient to achieve a desired effect in an individual being treated. For instance, this may be the amount necessary to inhibit or reduce the severity of cancer or proliferative disease in an individual.

As used herein, the term “Combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

### Arginase 1 Binders

The Arginase 1 binders of the present invention are human antibodies obtained from transgenic mice engineered to contain the entire human variable region repertoires, inserted by gene targeting at all three immunoglobulin loci (heavy, kappa, and lambda) in which expression of the human VH, VK and VL repertoire is regulated by mouse genomic regulatory sequences, and produces human VH and VL on mouse heavy and light chain constant domains. To produce human antibodies, the human VL and VL are transferred to human HC and LC constant domains. The Arginase 1 binder of the present invention comprises VH and VL derived from monoclonal antibody mAb5 obtained from a transgenic mouse immunized with hArg1.

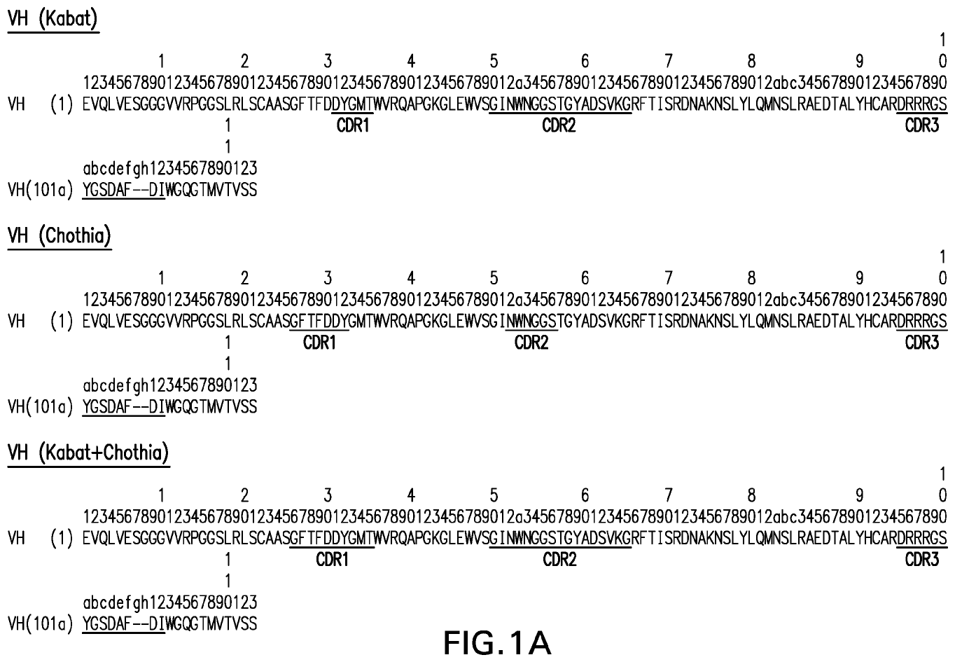

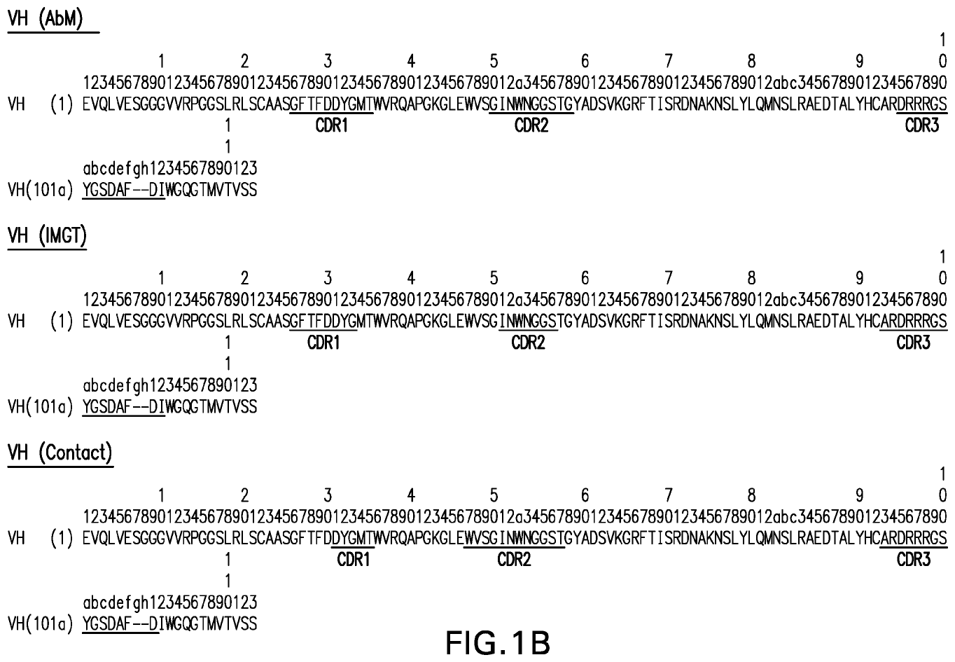

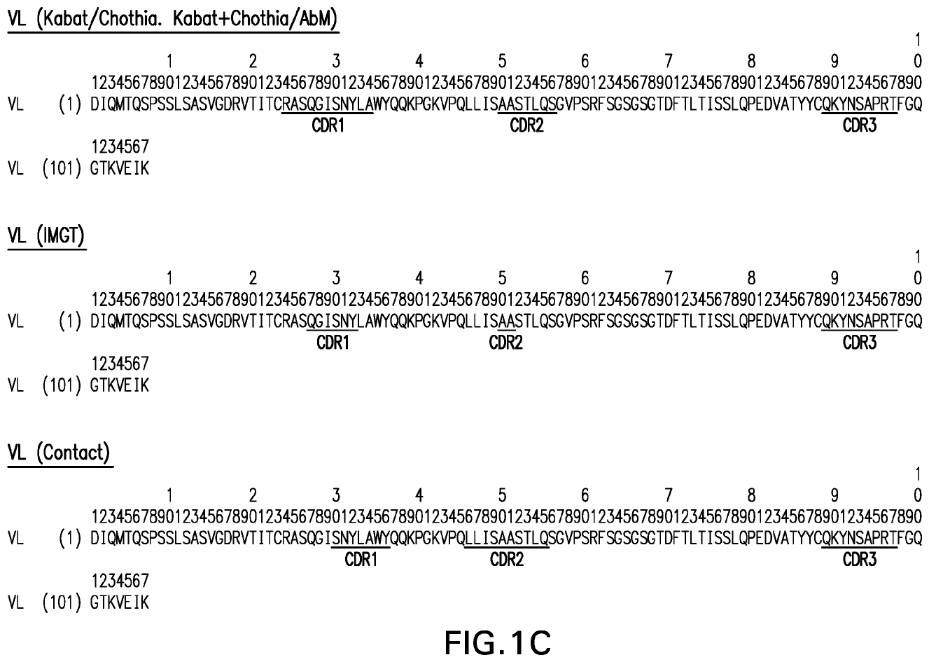

These Arginase 1 binders of the present invention comprise six complementarity determining regions (CDRs) comprising a particular combination of three CDRs as presented in the table below. The CDR amino acid sequences shown in Tables 2-7 and FIGS. 1A-1C are set forth according to the Kabat, Chothia, Kabat+Chothia, AbM. IMGT, and Contact numbering schemes for identifying CDR amino acid sequences. A particular CDR amino acid sequence determined using any one of the schemes advanced for identifying CDR amino acid sequences (See Table 1) have more or less amino acids than that of CDR amino acid sequences identified according to any other numbering scheme but will overlap the CDR amino acid sequences. Thus, the CDR amino acid sequences shown herein are not to be construed as limiting and any Arginase 1 binder in which the CDR amino acid sequences have been identified by another numbering scheme will fall within the scope of the Arginase 1 binders of the present invention provided the amino acid sequences for such Arginase 1 binders comprise the six CDR amino acid sequences as identified by any one of the numbering schemes and shown in Tables 2-7 and FIGS. 1A-1C. For all antibodies disclosed herein unless indicated otherwise, the amino acids comprising the variable domains are numbered according to the Kabat numbering scheme independently of how the amino acids are defined using Kabat. Chothia, Kabat+Chothia, AbM, IMGT, or Contact schemes and the constant domains are numbered according to the Eu numbering scheme.

The Arginase 1 binders comprise a VH and a VL, each domain comprising three CDRs and four Frameworks (FR) in the following arrangement

FR1-CDR1-FR2-CDR2-FR3-CDR3-FR4.

The Arginase 1 binder VH-CDRs and VL-CDRs may comprise the amino acid sequences shown in Tables 2-7 as defined according to Kabat, Chothia, Kabat+Chothia, AbM, IMGT, and Contact numbering schemes, respectively.

TABLE 2
Amino Acid Sequences of Arginase 1 Binder VH and VL CDRs
as defined by Kabat

| | VH-CDR1 | VH-CDR2 | VH-CDR3 |
|---|---|---|---|
| VH | DYMGT | GINWNGGSTGYADSVKG | DRRRGSYGSDAFDI |
| | (SEQ IF NO: 4) | (SEQ ID NO: 5) | (SEQ ID NO: 6) |
| | VL-CDR1 | VL-CDR2 | VL-CDR3 |
| VL | RASQGISNYLA | AASTLQS | QKYNSAPRT |
| | (SEQ ID NO: 17) | (SEQ ID NO: 18) | (SEQ ID NO: 19) |

TABLE 3
Amino Acid Sequences of Arginase 1 Binders VH and VL CDRs
as defined by Chothia

| | VH-CDR1 | VH-CDR2 | VH-CDR3 |
|---|---|---|---|
| VH | GFTFDDY | NWNGGS | DRRRGSYGSDAFDI |
| | (SEQ IF NO: 7) | (SEQ ID NO: 8) | (SEQ ID NO: 6) |
| | VL-CDR1 | VL-CDR2 | VL-CDR3 |
| VL | RASQGISNYLA | AASTLQS | QKYNSAPRT |
| | (SEQ ID NO: 17) | (SEQ ID NO: 18) | (SEQ ID NO: 19) |

TABLE 4
Amino Acid Sequences of Arginase 1 Binders VH and VL CDRs
as defined by Kabat+ Chothia

| | VH-CDR1 | VH-CDR2 | VH-CDR3 |
|---|---|---|---|
| VH | GFTFDDYGMT | GINWNGGSTGYADSVKG | DRRRGSYGSDAFDI |
| | (SEQ IF NO: 9) | (SEQ ID NO: 5) | (SEQ ID NO: 6) |
| | VL-CDR1 | VL-CDR2 | VL-CDR3 |
| VL | RASQGISNYLA | AASTLQS | QKYNSAPRT |
| | (SEQ ID NO: 17) | (SEQ ID NO: 18) | (SEQ ID NO: 19) |

TABLE 5
Amino Acid Sequences of Arginase 1 Binders VH and VL CDRs
as defined by AbM

| | VH-CDR1 | VH-CDR2 | VH-CDR3 |
|---|---|---|---|
| VH | GFTFDDYGMT | GINWNGGSTG | DRRRGSYGSDAFDI |
| | (SEQ IF NO: 9) | (SEQ ID NO: 10) | (SEQ ID NO: 6) |
| | VL-CDR1 | VL-CDR2 | VL-CDR3 |
| VL | RASQGISNYLA | AASTLQS | QKYNSAPRT |
| | (SEQ ID NO: 17) | (SEQ ID NO: 18) | (SEQ ID NO: 19) |

TABLE 6
Amino Acid Sequences of Arginase 1 Binders VH and VL CDRs
as defined by IMGT

| | VH-CDR1 | VH-CDR2 | VH-CDR3 |
|---|---|---|---|
| VH | GFTFDDYG | RRGSYGSDAFDI | ARDRRRGSYGSDAFDI |
| | (SEQ IF NO: 11) | (SEQ ID NO: 12) | (SEQ ID NO: 13) |
| | VL-CDR1 | VL-CDR2 | VL-CDR3 |
| VL | QGISNY | AA | QKYNSAPRT |
| | (SEQ ID NO: 20) | | (SEQ ID NO: 19) |

TABLE 7
Amino Acid Sequences of Arginase 1 Binders VH and VL CDRs
as defined by Contact

| | VH-CDR1 | VH-CDR2 | VH-CDR3 |
|---|---|---|---|
| VH | DDYGMT | WVSGINWNGGSTG | ARDRRRGSYGSDAFD |
| | (SEQ IF NO: 14) | (SEQ ID NO: 15) | (SEQ ID NO: 16) |
| | VL-CDR1 | VL-CDR2 | VL-CDR3 |
| VL | SSYLAWY | LLIYDASNRA | QQHSLLPR |
| | (SEQ ID NO: 21) | (SEQ ID NO: 22) | (SEQ ID NO: 23) |

In particular embodiments of the invention, the Arginase 1 binder comprises (a) a VH-CDR1 comprising the amino acid sequence set forth in SEQ ID NO: 4, a VH-CDR2 comprising the amino acid sequence set forth in SEQ ID NO: 5, and a VH-CDR3 comprising the amino acid sequence set forth in SEQ ID NO: 6; and (b) a VL-CDR1 comprising the amino acid sequence set forth in SEQ ID NO: 17, a VL-CDR2 comprising the amino acid sequence set forth in SEQ ID NO: 18, and a VL-CDR3 comprising the amino acid sequence set forth in SEQ ID NO: 19. The Arginase 1 binders specifically bind arginase 1 and inhibit arginase 1 activity. CDR sequences are determined using the Kabat numbering scheme.

In particular embodiments of the invention, the Arginase 1 binder comprises (a) a VH-CDR1 comprising the amino acid sequence set forth in SEQ ID NO: 7, a VH-CDR2 comprising the amino acid sequence set forth in SEQ ID NO: 8, and a VH-CDR3 comprising the amino acid sequence set forth in SEQ ID NO: 6; and (b) a VL-CDR1 comprising the amino acid sequence set forth in SEQ ID NO: 17, a VL-CDR2 comprising the amino acid sequence set forth in SEQ ID NO: 18, and a VL-CDR3 comprising the amino acid sequence set forth in SEQ ID NO: 19. The Arginase 1 binders specifically bind arginase 1 and inhibit arginase 1 activity. CDR sequences are determined using the Chothia numbering scheme.

In particular embodiments of the invention, the Arginase 1 binder comprises (a) a VH-CDR1 comprising the amino acid sequence set forth in SEQ ID NO: 9, a VH-CDR2 comprising the amino acid sequence set forth in SEQ ID NO: 5, and a VH-CDR3 comprising the amino acid sequence set forth in SEQ ID NO: 6. and (b) a VL-CDR1 comprising the amino acid sequence set forth in SEQ ID NO: 17, a VL-CDR2 comprising the amino acid sequence set forth in SEQ ID NO: 18, and a VL-CDR3 comprising the amino acid sequence set forth in SEQ ID NO: 19. The Arginase 1 binders specifically bind arginase 1 and inhibit arginase 1 activity. CDR sequences are determined using the Kabat+Chothia numbering scheme.

In particular embodiments of the invention, the Arginase 1 binder comprises (a) a VH-CDR1 comprising the amino acid sequence set forth in SEQ ID NO: 9, a VH-CDR2 comprising the amino acid sequence set forth in SEQ ID NO: 10, and a VH-CDR3 comprising the amino acid sequence set forth in SEQ ID NO: 6: and (b) a VL-CDR1 comprising the amino acid sequence set forth in SEQ ID NO: 17, a VL-CDR2 comprising the amino acid sequence set forth in SEQ ID NO: 18, and a VL-CDR3 comprising the amino acid sequence set forth in SEQ ID NO: 19. The Arginase 1 binders specifically bind arginase 1 and inhibit arginase 1 activity. CDR sequences are determined using the AbM numbering scheme.

In particular embodiments of the invention, the Arginase 1 binder comprises (a) a VH-CDR1 comprising the amino acid sequence set forth in SEQ ID NO: 11, a VH-CDR2 comprising the amino acid sequence set forth in SEQ ID NO: 12, and a VH-CDR3 comprising the amino acid sequence set forth in SEQ ID NO: 13: and (b) a VL-CDR1 comprising the amino acid sequence set forth in SEQ ID NO: 20, a VL-CDR2 comprising the amino acid sequence AA, and a VL-CDR3 comprising the amino acid sequence set forth in SEQ ID NO: 19. The Arginase 1 binders specifically bind arginase 1 and inhibit arginase 1 activity. CDR sequences are determined using the IMGT numbering scheme.

In particular embodiments of the invention, the Arginase 1 binder comprises (a) a VH-CDR1 comprising the amino acid sequence set forth in SEQ ID NO: 14, a VH-CDR2 comprising the amino acid sequence set forth in SEQ ID NO: 15, and a VH-CDR3 comprising the amino acid sequence set forth in SEQ ID NO. 16: and (b) a VL-CDR1 comprising the amino acid sequence set forth in SEQ ID NO: 21, a VL-CDR2 comprising the amino acid sequence set forth in SEQ ID NO: 22, and a VL-CDR3 comprising the amino acid sequence set forth in SEQ ID NO: 23. The Arginase 1 binders specifically bind arginase 1 and inhibit arginase 1 activity. CDR sequences are determined using the Contact numbering scheme.

In further embodiments, the Arginase 1 binder comprises a VH comprising the amino acid sequence set forth for VH in SEQ ID NO: 2 and a VL comprising the amino acid sequence set forth in SEQ ID NO: 3.

In further embodiments of the invention, the Arginase 1 binder comprises a heavy chain constant domain of the IgG1, IgG2, IgG3, or lgG4 isotype. In particular embodiments, the heavy chain constant domain comprises 1, 2, 3. 4, 5, 6, 7, 8, 9, or 10 amino acid substitutions, additions, deletions, or combinations thereof compared to the amino acid sequence of the native IgG1, IgG2, IgG3, or lgG4 isotype, wherein the Arginase 1 binder specifically binds arginase 1 and inhibits arginase 1 activity.

In further embodiments, the arginase binder inhibits arginase 1 activity by at least 50%, 60%, 70%, 80%, or 90%. In further embodiments, the arginase binder inhibits arginase 1 activity at an IC₅₀ of less than 100 nM, 50 nM, 20 nM, or 10 nM. In a further embodiment, the IC₅₀ is about 3.3 +/−0.3 nM or about 5.3 +/−0.8 nM.

In further embodiments of the invention, the Arginase 1 binder comprises a heavy chain constant domain of the human IgG1 or IgG4 isotype. In particular embodiments, the heavy chain constant domain comprises 1, 2, 3, 4, 5, 6, 7, 8, 9, or 10 amino acid substitutions, additions, deletions, or combinations thereof compared to the amino acid sequence of the native IgG1 or IgG4 isotype, wherein the Arginase 1 binder specifically binds arginase 1 and inhibits arginase 1 activity. In a further aspect, the heavy chain constant domain is of the IgG4 isotype and further includes a substitution of the serine residue at position 228 (EU numbering) with proline, which corresponds to position 108 of SEQ ID NO: 39 (Serine at position 108).

In further aspects or embodiments of the invention, the antibody comprises a IgG1 heavy chain constant domain comprising the amino acid sequence shown in SEQ ID NO: 24 or a variant thereof comprising 1, 2, 3, 4, 5, 6, 7, 8, 9, or 10 amino acid substitutions, additions. deletions, or combinations thereof, wherein the Arginase 1 binder specifically binds arginase 1 and inhibits arginase 1 activity.

In further aspects or embodiments of the invention, the antibody comprises a IgG2 heavy chain constant domain comprising the amino acid sequence shown in SEQ ID NO: 32 or a variant thereof comprising 1, 2, 3, 4, 5, 6, 7, 8, 9, or 10 amino acid substitutions, additions. deletions, or combinations thereof, wherein the Arginase 1 binder specifically binds arginase 1 and inhibits arginase 1 activity.

In further aspects or embodiments of the invention, the antibody comprises a IgG4 heavy chain constant domain comprising the amino acid sequence shown in SEQ ID NO: 39 and further comprising 1, 2, 3, 4, 5, 6, 7, 8, 9, or 10 amino acid substitutions, additions, deletions, or combinations thereof, wherein the Arginase 1 binder specifically binds arginase 1 and inhibits arginase 1 activity.

In particular embodiments of the invention, the constant domains as disclosed herein may comprise a C-terminal lysine or lack either a C-terminal lysine or a C-terminal glycine-lysine dipeptide.

In any one of the embodiments disclosed herein the light chain may comprise a human kappa light chain constant domain comprising SEQ ID NO: 44 or a lambda light chain constant domain comprising SEQ ID NO: 47.

### Arginase 1 Binders Comprising an Effector-Silent Fc Domain

Effector-silent Arginase 1 binders of the present invention are antibodies that comprise an HC constant domain or Fc domain thereof that has been modified such that the antibody displays no measurable binding to one or more FcRs or displays reduced binding to one or more FcRs compared to that of an unmodified antibody of the same IgG isotype. The effector-silent antibodies may in further embodiments display no measurable binding to each of FcγRIIIa. FcγRIIa, and FcγRI or display reduced binding to each of FcγRIIIa, FcγRIIa, and FcγRI compared to that of an unmodified antibody of the same IgG isotype. In particular embodiments, the HC constant domain or Fc domain is a human HC constant domain or Fc domain.

In particular embodiments, the effector-silent antibody comprises an Fc domain of an IgG1 or IgG2, IgG3, or IgG4 isotype that has been modified to lack N-glycosylation of the asparagine (Asn) residue at position 297 (Eu numbering system) of the HC constant domain. The consensus sequence for N-glycosylation is Asn-Xaa-Ser/Thr (wherein Xaa at position 298 is any amino acid except Pro); in all four isotypes the N-glycosylation consensus sequence is Asn-Ser-Thr. The modification may be achieved by replacing the codon encoding the Asn at position 297 in the nucleic acid molecule encoding the HC constant domain with a codon encoding another amino acid, for example Ala, Asp, Gln, Gly, or Glu, e.g. N297A, N297Q, N297G, N297E, or N297D. Alternatively, the codon for Ser at position 298 may be replaced with the codon for Pro or the codon for Thr at position 299 may be replaced with any codon except the codon for Ser. In a further alternative each of the amino acids comprising the N-glycosylation consensus sequence is replaced with another amino acid. Such modified IgG molecules have no measurable effector function. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations. In further embodiments, such IgGs modified to lack N-glycosylation at position 297 may further include one or more additional mutations disclosed herein for eliminating measurable effector function.

An exemplary IgG1 HC constant domain mutated at position 297, which abolishes the N-glycosylation of the HC constant domain, is set forth in SEQ ID NO: 31, an exemplary IgG2 HC constant domain mutated at position 297, which abolishes the N-glycosylation of the HC constant. is set forth in SEQ ID NO: 37. and an exemplary IgG4 HC constant domain mutated at position 297 to abolish N-glycosylation of the HC constant domain is set forth in SEQ ID NO: 43. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the Fc domain of the IgG1 IgG2, IgG3, or IgG4 HC constant domain comprising the effector-silent antibody is modified to include one or more amino acid substitutions selected from E233P, L234A, L235A, L235E, N297A, N297D, D265S, and P331S (wherein the positions are identified according to Eu numbering) and wherein said HC constant domain is effector-silent. In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the HC constant domain comprises L234A, L235A, and D265S substitutions (wherein the positions are identified according to Eu numbering). In particular embodiments, the HC constant domain comprises an amino acid substitution at position Pro329 and at least one further amino acid substitution selected from E233P, L234A, L235A, L235E, N297A, N297D, D265S, and P331S (wherein the positions are identified according to Eu numbering). These and other substitutions are disclosed in WO9428027; WO2004099249; WO20121300831, U.S. Pat. Nos. 9,708,406; 8,969.526; 9,296,815; Sondermann et al. Nature 406, 267-273 (2000)).

In particular embodiments of the above, the HC constant domain comprises an L234A/L235A/D265A; L234A/L235A/P329G; L235E; D265A; D265A/N297G; or V234A/G237A/P238S/H268A/V309L/A330S/P331S substitutions, wherein the positions are identified according to Eu numbering. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibody comprises an IgG1 isotype, in which the Fc domain of the HC constant domain has been modified to be effector-silent by substituting the amino acids from position 233 to position 236 of the IgG1 with the corresponding amino acids of the human IgG2 HC and substituting the amino acids at positions 327, 330, and 331 with the corresponding amino acids of the human IgG4 HC, wherein the positions are identified according to Eu numbering (Armour et al., Eur. J. Immunol. 29(8):2613-24 (1999); Shields et al., J. Biol. Chem. 276(9):6591-604(2001)). In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibody comprises a VH fused or linked to a hybrid human immunoglobulin HC constant domain, which includes a hinge region, a CH2 domain and a CH3 domain in an N-terminal to C-terminal direction, wherein the hinge region comprises an at least partial amino acid sequence of a human IgD hinge region or a human IgG1 hinge region: and the CH2 domain is of a human IgG4 CH2 domain, a portion of which, at its N-terminal region, is replaced by 4-37 amino acid residues of an N-terminal region of a human IgG2 CH2 or human IgD CH2 domain. Such hybrid human HC constant domain is disclosed in U.S. Pat. No. 7,867,491, which is incorporated herein by reference in its entirety.

In particular embodiments, the effector-silent antibody comprises an IgG4 HC constant domain in which the serine at position 228 according to the Eu system is substituted with proline, see for example SEQ ID NO: 39. This modification prevents formation of a potential inter-chain disulfide bond between the cysteines at positions Cys226 and Cys229 in the EU numbering scheme and which may interfere with proper intra-chain disulfide bond formation. See Angal et al. Mol. Imunol. 30:105 (1993): see also (Schuurman et al., Mol. Immunol. 38: 1-8, (2001)). In further embodiments, the IgG4 constant domain includes in addition to the S228P substitution, a P239G, D265A, or D265A/N297G amino acid substitution, wherein the positions are identified according to Eu numbering. In particular embodiments of the above, the IgG4 HC constant domain is a human HC constant domain. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

Exemplary effector-silent IgG1 HC constant domains include HC constant domains comprising an amino acid sequence selected from the group consisting of amino acid sequences set forth in SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 45 and SEQ ID NO: 46. Exemplary effector-silent IgG2 HC constant domains have an amino acid sequence selected from the group consisting of amino acid sequences set forth in SEQ ID NO: 33, SEQ ID NO: 34. SEQ ID NO: 35, SEQ ID NO: 36, SEQ ID NO: 37. and SEQ ID NO: 38. Exemplary effector-silent IgG4 HC constant domains have an amino acid sequence selected from the group consisting of amino acid sequences set forth in SEQ ID NO: 39. SEQ ID NO: 40, SEQ ID NO: 41, SEQ ID NO: 42. and SEQ ID NO: 43.

In particular embodiments of the Arginase 1 binder , the Arginase 1 binder is an antibody comprising an IgG1, IgG2, or IgG4 Fc domain as disclosed herein, which further comprises a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 44 or a lambda light chain constant domain comprising SEQ ID NO: 47.

### Nucleic Acid Molecules Encoding the Arginase 1 Binders

The present invention further provides nucleic acid molecules that encode the Arginase 1 binders of the present invention. In particular embodiments, the Arginase 1 binder comprises a VH encoded by a nucleic acid molecule comprising the nucleotide sequence set forth in SEQ ID NO: 51. In a further embodiment, Arginase 1 binder further comprises a VL encoded by a nucleic acid molecule comprising the nucleotide sequence set forth in SEQ ID NO: 52.

In further embodiments, the Arginase 1 binder comprises a VH encoded by a nucleic acid molecule comprising the nucleotide sequence set forth in SEQ ID NO: 51 and a VL encoded by a nucleic acid molecule comprising the nucleotide sequence set forth in SEQ ID NO: 52.

In further embodiments, the Arginase 1 binder comprises a VH domain encoded by a nucleic acid molecule comprising the nucleotide sequence set forth in SEQ ID NO: 51 and a VL encoded by a nucleic acid molecule comprising the nucleotide sequence set forth in SEQ ID NO: 52, wherein the nucleic acid molecule encoding the VH is linked to a nucleic acid molecule encoding an IgG1, IgG2, IGG3, or IgG4 heavy chain constant domain and the nucleic acid molecule encoding the VL is linked to a nucleic acid molecule encoding kappa or lambda light chain constant domain.

Exemplary IgG1 heavy chain constant domains may be encoded by a nucleic acid molecule comprising the nucleotide sequence set forth in SEQ ID NO: 53, SEQ ID NO: 54, SEQ ID NO: 55, SEQ ID NO: 56, SEQ ID NO: 57, SEQ ID NO: 58. SEQ ID NO: 59, SEQ ID NO: 60, SEQ ID NO: 74, or SEQ ID NO: 75.

Exemplary IgG2 heavy chain constant domains may be encoded by a nucleic acid molecule comprising the nucleotide sequence set forth in SEQ ID NO: 61, SEQ ID NO: 62, SEQ ID NO: 63, SEQ ID NO: 64, SEQ ID NO: 65, SEQ ID NO: 66, or SEQ ID NO: 67.

Exemplary IgG4 heavy chain constant domains may be encoded by a nucleic acid molecule comprising the nucleotide sequence set forth in SEQ ID NO: 68, SEQ ID NO: 69, SEQ ID NO: 70, SEQ ID NO: 71, or SEQ ID NO: 72.

Exemplary light chain constant domains may be encoded by a nucleic acid molecule comprising the nucleotide sequence set forth in SEQ ID NO: 73 or SEQ ID NO: 76.

In particular embodiments, the HC and LC (or VH and VL) are expressed as a fusion protein in which the N-terminus of the HC and the LC (or VH and VL) are fused to a leader peptide to facilitate the transport of the antibody through the secretory pathway. Examples of leader peptides that may be used include MSVPTQVLGLLLLWLTDARC (SEQ ID NO: 77) encoded by the nucleotide sequence set forth in SEQ ID NO: 79 or MEWSWVFLFFLSVTTGVHS (SEQ ID NO: 78) encoded by the nucleotide sequence set forth in SEQ ID NO: 80. Thus, in particular embodiments, the aforementioned nucleic acid molecules may comprise a polynucleotide encoding a leader peptide linked to the 5′ end of the nucleic acid molecule.

The nucleic acid molecules disclosed herein may include one or more substitutions that optimize one or more of the codons for enhancing the expression of the nucleic acid molecule in a particular host cell, e.g., yeast or fungal host cell, non-human mammalian hot cell, human host cell, insect host cell, or prokaryote host cell. Methods and computer programs for optimizing a nucleic acid molecule for enhancing expression in a particular host cell are well known in the art, e.g. the IDT Codon Optimization Tool commercially available from Integrated DNA Technologies, Inc. 1710 Commercial Park, Coralville, Iowa 52241, USA.; U.S. Pat. No. 8,326,547; WO2020024917A1.

### Methods for Making Arginase 1 Binders

The present invention includes recombinant methods for making Arginase 1 binders comprising introducing into a host cell (i) an expression vector that encodes the VH and VL of an Arginase 1 binder or the HC and LC of an Arginase 1 binder, or (ii) two expression vectors, one encoding the VH of an Arginase 1 binder or the HC of an Arginase 1 binder the other encoding the VL of an Arginase 1 binder or the LC of an Arginase 1 binder. The nucleic acid molecules or polynucleotides encoding the VH, VL, HC, or LC are operably linked to a promoter and other transcription and translation regulatory sequences. The host cell is cultured under conditions and a time period suitable for expression of the nucleic acid molecules followed by isolating the Arginase 1 binder from the host cell and/or medium in which the host cell is grown. See e.g., WO 04/041862, WO 2006/122786, WO 2008/020079, WO 2008/142164 or WO 2009/068627. The expression vector may be a plasmid or viral vector. The invention also relates to hosts or host cells that contain such nucleic acid molecule encoding the Arginase 1 binders or components thereof, e.g., solely the VH or HC or solely the VL or LC.

Eukaryotic and prokaryotic host cells, including mammalian cells as hosts for expression of the Arginase 1 binder are well known in the art and include many immortalized cell lines available from the American Type Culture Collection (ATCC). These include, but are not limited to, Chinese hamster ovary (CHO) cells, NSO, SP2 cells, HeLa cells. baby hamster kidney (BHK) cells, monkey kidney cells (COS), human hepatocellular carcinoma cells (e.g., Hep G2), A549 cells, 3T3 cells, HEK-293 cells and a number of other cell lines. Thus, mammalian host cells include human, mouse, rat, dog, monkey, pig, goat, bovine, horse and hamster cells. Cell lines of particular preference are selected through determining which cell lines have high expression levels. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Saccharomyces cervisiea, and Trichoderma reesei. The present invention includes any host cell comprising an Arginase 1 binder of the present invention or comprising one or more nucleic acid molecules encoding such an Arginase 1 binder or comprising an expression vector that comprises one or more nucleic acid molecules encoding such Arginase 1 binder.

Further, expression of an Arginase 1 binder from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4. Thus, in an embodiment of the invention, the mammalian host cells lack a glutamine synthetase gene and are grown in the absence of glutamine in the medium wherein, however, the nucleic acid molecule encoding the immunoglobulin chain comprises a glutamine synthetase gene which complements the lack of the gene in the host cell. Such host cells containing the Arginase 1 binder or nucleic acid(s) or expression vector(s) as discussed herein as well as expression methods, as discussed herein, for making the Arginase 1 binder using such a host cell are part of the present invention.

The present invention includes methods for purifying an Arginase 1 binder comprising introducing a sample (e.g., culture medium, cell lysate or cell lysate fraction, e.g., a soluble fraction of the lysate) comprising the Arginase 1 binder to a purification medium (e.g., cation-exchange medium, anion-exchange medium and/or hydrophobic exchange medium) and either collecting purified Arginase 1 binder from the flow-through fraction of said sample that does not bind to the medium; or, discarding the flow-through fraction and eluting bound Arginase 1 binder from the medium and collecting the eluate. In an embodiment of the invention, the medium is in a column to which the sample is applied. In an embodiment of the invention, the purification method is conducted following recombinant expression of the Arginase 1 binder in a host cell, e.g., wherein the host cell is first lysed and, optionally, the lysate is purified of insoluble materials prior to purification on a medium: or wherein the Arginase 1 binder is secreted into the culture medium by the host cell and the medium or a fraction thereof is applied to the purification medium.

In general, glycoproteins produced in a particular cell line or transgenic animal will have a glycosylation pattern that is characteristic for glycoproteins produced in the cell line or transgenic animal. Therefore, the particular glycosylation pattern of an Arginase 1 binder will depend on the particular cell line or transgenic animal used to produce the Arginase 1 binder. Arginase 1 binders comprising only non-fucosylated N-glycans are part of the present invention and may be advantageous, because non-fucosylated antibodies have been shown to typically exhibit more potent efficacy than their fucosylated counterparts both in vitro and in vivo (See for example. Shinkawa et al., J. Biol. Chem. 278: 3466-3473 (2003); U.S. Pat. Nos. 6.946.292 and 7,214,775). These Arginase 1 binders with non-fucosylated N-glycans are not likely to be immunogenic because their carbohydrate structures are a normal component of the population that exists in human serum IgG.

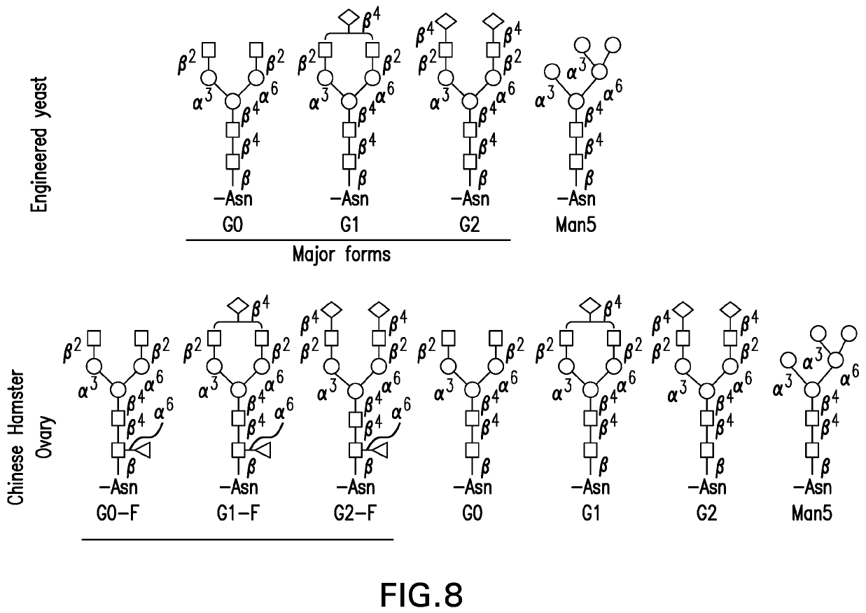

The present invention includes Arginase 1 binders comprising N-linked glycans that are typically added to immunoglobulins produced in Chinese hamster ovary cells (CHO N-linked glycans) or to engineered yeast cells (engineered yeast N-linked glycans), such as, for example. Pichia pastoris. For example, in an embodiment of the invention, the Arginase 1 binder comprises one or more of the “engineered yeast N-linked glycans” or “CHO N-linked glycans” (e.g., G0 and/or G0-F and/or G1 and/or G1-F and/or and/or G2-F and/or Man5. see FIG. 8). In an embodiment of the invention, the Arginase 1 binder comprises the engineered yeast N-linked glycans, i.e., G0 and/or G1 and/or G2, optionally, further including Man5. In an embodiment of the invention, the Arginase 1 binders comprise the CHO N-linked glycans, i.e., G0-F, G1-F and G2-F, optionally, further including G0 and/or G1 and/or G2 and/or Man5. In an embodiment of the invention, about 80% to about 95% (e.g., about 80-90%, about 85%, about 90% or about 95%) of all N-linked glycans on the Arginase 1 binders are engineered yeast N-linked glycans or CHO N-linked glycans. See Nett et al. Yeast. 28(3): 237-252 (2011); Hamilton et al. Science. 313(5792): 1441-1443 (2006); Hamilton et al. Curr Opin Biotechnol. 18(5): 387-392 (2007). For example, in an embodiment of the invention, an engineered yeast cell is GFI5.0 or YGLY8316 or strains set forth in U.S. Pat. No. 7,795,002 or Zha et al. Methods Mol Biol. 988:31-43 (2013). See also international patent application publication no. WO2013/066765.

### Administration Pharmaceutical Compositions

The Arginase 1 binder may be provided in suitable pharmaceutical compositions comprising the Arginase 1 binder and a pharmaceutically acceptable carrier. The carrier may be diluent, adjuvant, excipient, or vehicle with which the Arginase 1 binder is administered. Such vehicles may be liquids, such as water and oils, including those of petroleum, animal, vegetable or synthetic origin, such as peanut oil, soy bean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins. e.g., human serum albumin, are described, for example, in e.g. Remington: The Science and Practice of Pharmacy, 21.sup. st Edition, Troy, D. B. ed., Lipincott Williams and Wilkins, Philadelphia, Pa. 2006, Part 5, Pharmaceutical Manufacturing pp 691-1092, see especially pp. 958-989.

The mode of administration of the Arginase 1 binder may be any suitable route such as parenteral administration, e.g., intradermal, intramuscular, intraperitoneal, intravenous or subcutaneous, pulmonary, transmucosal (oral, intranasal, intravaginal, rectal) or other means appreciated by the skilled artisan, as well known in the art.

The Arginase 1 binder may be administered to an individual (e.g., patient) by any suitable route, for example parentally by intravenous (i.v.) infusion or bolus injection, intramuscularly or subcutaneously, or intraperitoneally. i.v. infusion may be given over for, example, 15, 30, 60, 90, 120, 180, or 240 minutes, or from 1, 2, 3, 4, 5, 6, 7, 8, 9, 10, 11 or 12 hours.

The dose given to an individual having cancer or malignancy is sufficient to alleviate or at least partially arrest the disease being treated (“therapeutically effective amount”) and may be sometimes 0.005 mg/kg to about 100 mg/kg, e.g. about 0.05 mg/kg to about 30 mg/kg or about 5 mg to about 25 mg/kg, or about 4 mg/kg, about 8 mg/kg, about 16 mg/kg or about 24 mg/kg, or, e.g., about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat cancer or malignancy, but 9, 10, 11, 12, 13, 14, 15, 16, 17, 18, 19, 20 or more doses may be given.

The administration of the Arginase 1 binder may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the Arginase 1 binder in the methods of the invention may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

The Arginase 1 binder may be administered by maintenance therapy, such as, e.g., once a week for a period of 6 months or more. For example, Arginase 1 binder in the methods of the invention may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The Arginase 1 binder may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The Arginase 1 binder may be lyophilized for storage and reconstituted in a suitable carrier prior to use. This technique has been shown to be effective with conventional protein preparations and well known lyophilization and reconstitution techniques can be employed.

### Combination Therapy Treatments

The combination therapy of the present invention comprises an Arginase 1 binder and another therapeutic agent (small molecule or antibody) may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy of the present invention may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

### Combination Therapy Comprising an Arginase 1 Binder and a Chemotherapy Agent

The combination therapy of the present invention may be administered to an individual having a cancer in combination with chemotherapy. The individual may undergo the chemotherapy at the same time the individual is undergoing the combination therapy of the present invention. The individual may undergo the combination therapy of the present invention after the individual has completed chemotherapy. The individual may be administered the chemotherapy after completion of the combination therapy. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
(xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See. e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608: Milgrom et al. (1999) New Engl. J. Med. 341: 1966-1973: Slamon et al. (2001) New Engl. J. Med. 344:783-792; Beniaminovitz et al. (2000) New Engl. J. Med. 342:613-619: Ghosh et al. (2003) New Engl. J. Med. 348:24-32; Lipsky et al. (2000) New Engl. J. Med. 343: 1594-1602: Physicians' Desk Reference 2003 (Physicians' Desk Reference, 57th Ed): Medical Economics Company; ISBN: 1563634457: 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

Thus, the present invention contemplates embodiments of the combination therapy of the present invention that further includes a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy further in combination with a chemotherapy step may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy with a chemotherapy step may be used to treat one or more cancers selected from melanoma (metastatic or unresectable), primary mediastinal large B-cell lymphoma (PMBCL), urothelial carcinoma. MSIHC, gastric cancer, cervical cancer, hepatocellular carcinoma (HCC), Merkel cell carcinoma (MCC), renal cell carcinoma (including advanced), and cutaneous squamous carcinoma.

### Combination Therapy Comprising an Arginase 1 Binder and a Therapeutic Antibody

The Arginase 1 binder of the present invention may be administered in combination with one or more therapeutic agent, which is an antibody, for treatment of cancer or proliferative disease. The individual may undergo treatment with the therapeutic antibody at the same time the individual is undergoing the combination therapy of the present invention. The individual may undergo the combination therapy of the present invention after the individual has completed treatment with the therapeutic antibody. The individual may be administered the treatment with the therapeutic antibody after completion of the combination therapy. The combination therapy of the present invention may also be administered to an individual having recurrent or metastatic cancer with disease progression or relapse cancer and who is undergoing chemotherapy or who has completed chemotherapy. In particular embodiments, the therapeutic agent targets the programmed death 1 receptor or ligand., PD-1 and PD-L1, respectively.

Exemplary anti-PD-1 antibodies that may be used in a combination therapy with the Arginase 1 binder include any antibody that binds PD-1 and inhibits PD-1 from binding PD-L1. In a further embodiment, the exemplary anti-PD-1 antibody is selected from the group consisting of nivolumab, pembrolizumab, and cemiplimab-rwlc. Exemplary antibodies include the following anti-PD-1 antibodies and compositions comprising an anti-PD1 antibody and a pharmaceutically acceptable salt.

Pembrolizumab, also known as KEYTRUDA, lambrolizumab, MK-3475 or SCH-900475, is a humanized anti-PD-1 antibody described in U.S. Pat. No. 8,354.509 and WO2009/114335 and disclosed, e.g., in Hamid, et al., New England J. Med. 369 (2): 134-144 (2013).

Nivolumab, also known as OPDIVO, MDX-1106-04, ONO-4538, or BMS-936558, is a fully human IgG4 anti-PD-1 antibody described in WO2006/121168 and U.S. Pat. No. 8,008,449.

Cemiplimab-rwlc, also known as cemiplimab, LIBTAYO or REGN2810, is a recombinant human IgG4 monoclonal antibody that is described in WO2015112800 and U.S. Pat. No. 9,987,500.

In particular embodiments, the anti-PD-1 antibody comprises (i) a VH comprising the three HC-CDRs of pembrolizumab fused or linked to an effector-silent HC constant domain and (ii) a VL comprising the three LC-CDRs of pembrolizumab fused or linked to a LC kappa or lambda constant domain.

In particular embodiments, the anti-PD-1 antibody comprises (i) a VH comprising the three HC-CDRs of nivolumab fused or linked to an effector-silent HC constant domain and (ii) a VL comprising the three LC-CDRs of nivolumab fused or linked to a LC kappa or lambda constant domain.

In particular embodiments, the anti-PD-1 antibody comprises (i) a VH comprising the three HC-CDRs of cemiplimab-rwlc fused or linked to an effector-silent HC constant domain and (ii) a VL comprising the three LC-CDRs of nivolumab fused or linked to a LC kappa or lambda constant domain.

In particular embodiments, the anti-PD-1 antibody VH may be fused or linked to an IgG1, IgG2, IgG3, or IgG4 HC constant domain that is not currently linked to the particular VH or is linked to an IgG1, IgG2, IgG3, or IgG4 HC constant domain has been modified to include one or more mutations in the Fc domain that render the resulting anti-PD-1 antibody effecter-silent.

### Injection Device for Administering an Arginase 1 Binder

The present invention also provides an injection device comprising an Arginase 1 binder as set forth herein or a pharmaceutical composition thereof. An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the Arginase 1 binder or a pharmaceutical composition thereof), a needle for piecing skin and/or blood vessels for injection of the fluid: and a plunger for pushing the fluid out of the cylinder and through the needle bore. In an embodiment of the invention, an injection device that comprises an Arginase 1 binder or a pharmaceutical composition thereof is an intravenous (IV) injection device. Such a device includes the Arginase 1 binder or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the subject through the cannula or trocar/needle.

The Arginase 1 binder or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the Arginase 1 binder or a pharmaceutical composition thereof to a patient's body. External infusion pumps are medical devices that deliver the Arginase 1 binder or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### Kits Comprising an Arginase 1 Binder

Further provided are kits comprising one or more components that include, but are not limited to, an Arginase 1 binder, as discussed herein in association with one or more additional components including, but not limited to, a further therapeutic agent, as discussed herein. The Arginase 1 binder and/or the therapeutic agent can be formulated as a pure composition or in combination with a pharmaceutically acceptable carrier, in a pharmaceutical composition.

In one embodiment, the kit includes an Arginase 1 binder or a pharmaceutical composition thereof in one container (e.g., in a sterile glass or plastic vial) and a further therapeutic agent in another container (e.g., in a sterile glass or plastic vial).

In another embodiment, the kit comprises a combination of the invention, including an Arginase 1 binder or pharmaceutical composition thereof in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above. Thus, the present invention includes a kit comprising an injection device and the Arginase 1 binder, e.g., wherein the injection device includes Arginase 1 binder or wherein the Arginase 1 binder is in a separate vessel.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

The following examples are intended to promote a further understanding of the present invention.

### EXAMPLE 1

### Expression and Purification of hArg1

Full-length untagged hArg1 was expressed in E. coli BL21 (DE3) cells using superbroth media. Expression was induced with 1 mM Isopropyl β-d-1-thiogalactopyranoside (IPTG) at OD₆₀₀ of 0.8 and cells were grown for four hours at 37° C. Cell pellets were resuspended in lysis buffer (10 mM Tris pH 7.5, 5 mM MnCl₂, 2 mM beta-mercaptoethanol (BME), 1 mg/mL lysozyme), passed through a microfluidizer three times at 15,000 pounds per square inch (PSI) and the soluble fraction was clarified by centrifugation at 11,000×g. Clarified lysates were heat treated at 60° C. for 20 minutes. Heat treated lysates were passed through a HiTRAP-SP column (GE). Flow through containing hArg1 was diluted to about 40 mM NaCl and reloaded on another HiTRAP-SP column. hArg1 was eluted from the column using a linear gradient from 20 mM NaCl to 1 M NaCl. Pooled fractions were concentrated and loaded on a HiLoad Superdex 200 26/60 size exclusion column in 25 mM HEPES pH 7.3, 150 mM NaCl, 1 mM MnCl. Peak fractions were analyzed by SDS-PAGE, pooled and concentrated. Purification adapted from Strickland Acta Cryst. (2011). F67, 90-93.

### EXAMPLE 2

### Antibody Discovery and Optimization

De novo antibody discovery for identifying mAb5 was from a Trianni mouse from a campaign run against hArg1. Trianni mice produce chimeric antibodies having human variable domains and mouse constant domains. The antibodies were variable region sequenced and the variable heavy domain (VH) was cloned forward of a human IgG1 D265A constant domain encoded region of a pTT5 vector. The variable light domain (VL) was cloned forward of a human kappa encoded domain of a pTT5 vector. All proteins were analytically and verified by size-exclusion chromatography (SEC) and sodium dodecyl sulfate polyacrylamide gel electrophoresis (SDS-PAGE). The sequences and CDRs of the VH and VL are shown FIGS. 1A-1C.

### EXAMPLE 3

### Antibody Production

ExpiCHO-S cells growing in suspension were transfected with indicated antibody expression plasmids (HC+LC) using commercially available protocols and ExpiFectamine CHO reagents (Thermo-Fisher). In brief, cells were transfected day 0 using 1 μg total DNA (3:2 ratio LC:HC) per 1 mL cells at a density of 6 million cells per mL and a viability >95% measured using a Vi-Cell (Beckman-Coulter). On day one, ExpiCHO feed and enhancer were added and culture temperature was lowered to 32° C. On day five, a second EXPI-CHO feed was performed and cell viability was measured using a Vi-Cell (Beckman-Coulter). Cultures were harvested between day 8 and day 12 depending on a cell viability greater than 80%. Antibody was purified from clarified supernatant using Protein A chromatography (mAbSelect Sure LX, GE Healthcare). Protein A was incubated with the clarified supernatant overnight in 4° C. on a roller mixer. Resin was then collected from supernatant mixture and transferred into a column and washed with 10 column volumes (CV) of PBS. Elution of antibody was achieved using 20 mM sodium acetate, pH 3.5. One CV fractions were collected and tested by Bradford assay to determine presence of protein. In some cases, Protein A purification was followed by anion exchange chromatography (Capto Q, GE Healthcare). Purified antibodies were buffer exchanged into the final formulation buffer of 20 mM sodium acetate, 9% sucrose, pH 5.5. Purified antibody was checked for purity by reduced and non-reduced Capillary electrophoresis sodium dodecyl sulfate (CE-SDS) (Perkin-Elmer), concentration was measured by A280, and aggregate content was analyzed by size exclusion ultra performance liquid chromatography (SEC-UPLC) using a BEH200 UPLC- SEC analytical column (Waters corporation). Endotoxin was quantified using Endosafe® nexgen-MCS™(Charles River). Intact mass was confirmed via Synapt G2S QTOF or Xevo-TOF (Waters).

### EXAMPLE 4

### Antibody Potency and Mechanism of hArg1 Inhibition

To evaluate antibody potency and mechanism of hArg1 inhibition, the antibodies to be tested were diluted in assay buffer (50 mM Tris pH 7.5, 50 mM sodium chloride, 1 mM MnCl, and 0.05% bovine serum albumin) to a concentration 2.5-fold higher than desired assay concentrations. To each well of a Greiner black 384-well assay plate (catalog #781086) was added 10 μL of antibody solution followed by 10 μL of assay buffer or assay buffer with 1 nM human or mouse Arg1. After 30 minutes of incubation at room temperature, 5 μL of a 5× solution of thioarginine (variable concentrations) was added. The assay was allowed to proceed for 60 minutes then quenched by addition of 15 μL of a solution of 375 μM 7-Diethylamine-3-(4-maleimidophenyl)-4-methylcoumarin (Sigma Chemical) in 70% ethanol to quench the reaction and detect thioornithine. The plate was briefly shaken to mix and the fluorescence was measured in a Spectramax plate reader (Molecular Devices) with a 410 nm excitation wavelength and an 490 nm emission wavelength. Kinetic data were fit to various models of enzyme inhibition (competitive, mixed, noncompetitive, and uncompetitive) using GraphPad Prism.

An alternate assessment of antibody potency was performed by serially diluting antibodies in assay buffer and performing the assay described above except that a fixed concentration of 1 mM thioarginine was used. Data were normalized to wells containing either no antibody (no effect control) or no arginine (max effect control). The percent inhibition was then fit to a four-parameter logistic equation in GraphPad Prism to determine the IC50.

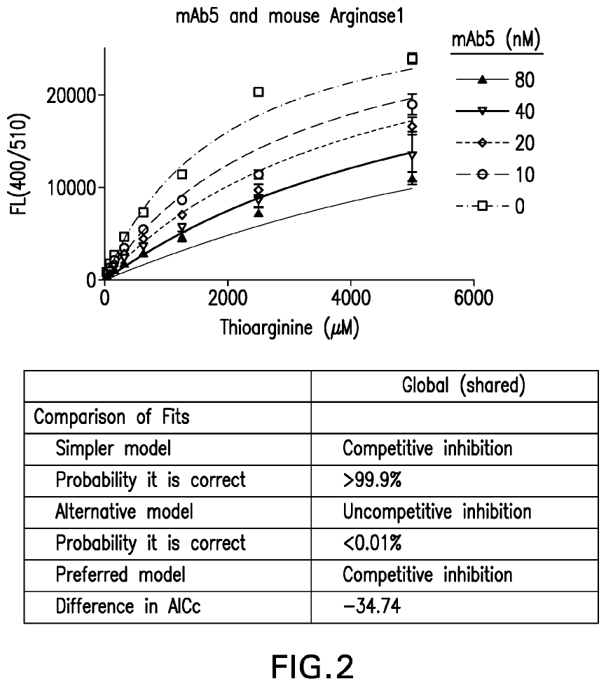

Potency and MOI for mAb5 is shown in FIG. 2.

### EXAMPLE 5

### Determination of hArg1 Activity by Liquid Chromatography-Mass Spectroscopy (LC-MS)

Arginase enzyme was diluted to 1.25 nM in assay buffer containing 50 mM Tris pH 7.5, 50 mM NaCl, 1 mM MnCl₂ and 0.05% Bovine serum albumin and 20 μL of the dilution were added to black 384 well assay plates (Greiner Bio-One Inc., Monroe, CA). Titrations of antibodies were added to appropriate wells on the plate, following which samples were incubated at 37° C. for 30 minutes in an oven. The arginine substrate was diluted in assay buffer to 1.5 mM and 5 μL were added into assay plate (final substrate concentration=300 μM). The reaction was carried out for one hour at 37° C. At the end of the reaction, 15 μL of ethanol containing internal standards (40 μM [¹³C6]-Arginine and [¹³C5]-Ornithine) were added to the mixture to quench the reaction. The assay plate was then stored at −80° C. until sample derivatization.

Then, 10 μL of thawed sample were transferred from the assay plate to a 384 deep-well plate and 90 μL methanol were added. Samples were centrifuged at 2,500 rpm for 10 minutes at room temperature and five μL of supernatant were transferred to a fresh 384 well SiliGuard-coated plate containing 35 μL of neat borate buffer. AccQ·Tag Ultra reagent (Waters Corporation, Milford, MA) was initially prepared according to the manufacturer's instructions and then diluted an additional two-fold with acetonitrile. Then, 10 mL of the reconstituted AccQ·Tag were added to each well and plates were sealed with pierceable aluminum foil (Agilent Technologies, Santa Clara, CA). The derivatization reaction was carried out by incubating at 55° C. in an oven for 30 minutes. Plates were then stored at 4° C. prior to LC-MS analysis.

Samples were analyzed on a Thermo TSQ Vantage triple quadrupole mass spectrometer with an electrospray ionization source operating in the positive ion mode (Thermo Fisher Scientific: Waltham, MA). Separation was achieved using an Acquity UPLC HSS-T3 2.1×30 mm, 1.8 μM column (Waters Corporation, Milford, MA) at ambient temperature. A binary solvent system composed of 0.1% formic acid in H2O (mobile phase A) and 0.1% formic acid in acetonitrile (mobile phase B) was used for chromatographic separation. Selected reaction monitoring was used to quantify the analytes and internal standards, with the specific precursor to product transitions of: Arginine (344.96>70.14), [¹³C6]Arginine (351.102>74.3), Ornithine (473.29>171.15), [¹³C5]Ornithine (478.29>171.15). During analysis, samples were maintained at 10° C.in the autosampler chamber. For injection, 12 μL of sample were over-filled into a 5 μL sample loop. All results were analyzed using the response ratio of analyte peak area/internal standard peak area for data normalization.

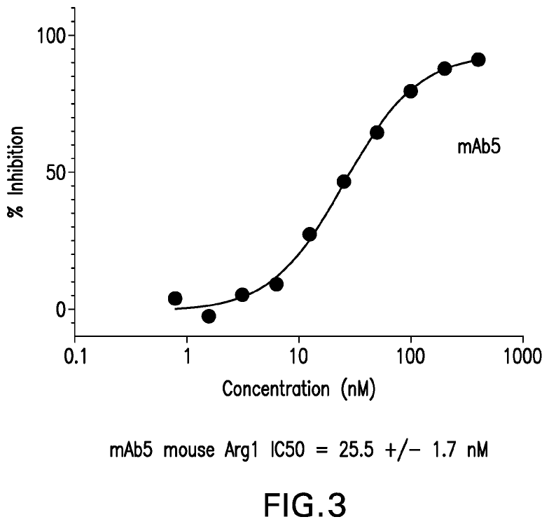

FIG. 3 shows dose response curves for mAb5 as determined by LC-MS.

### EXAMPLE 6

### Antibody Binding Affinities to Trimeric hArg1 and Monomeric hArg1

The antibodies have interactions spanning across the hArg1 monomeric interfaces when hArg1 is present in the natural, trimeric form. SPR assays revealed the reduction or loss of binding potencies of the mAbs when hArg1 was forced into a monomeric state.

The surface area between hArg1 and mAb5 HC and LC were as follows: monomer A shares 634 Å² and 5 salt bridges with the HC and shares 274 Å² and no salt bridge (Table 8).

TABLE 8
Surface area, hydrogen bonds, and
salt bridges between antibodies and hArg1

| | hArg1 | Antibody | Surface area | # Hydrogen | # salt |
|---|---|---|---|---|---|
| Antibody | monomer | chain | (Å²) | bonds | bridges |
| mAb5 | MonA | HC | 634 | 8 | 5 |
| | MonA | LC | 274 | 3 | 0 |

SA calcs done with PISA (Krissinel & Henrick, J. Mol. Biol. 372, 774-797 (2007))

### EXAMPLE 7

### Cryo-Electron Microscopy Methods and Image Processing

All the hArg1:mAb complexes were formed by mixing the protein and the mAb in reaction buffer (25 mM HEPES pH 7.4. 150 mM NaCl, 1.0 mM MnCl2) at 3:1 molar ratio and incubating the mixture for 30 minutes before preparing the grids. Grids were prepared and data were collected at NanoImaging Services (San Diego, CA) according to the following protocol and the specifications in Tables 9 and 10: After incubation, the sample containing the complex was diluted with reaction buffer to approx. Then, 75 μM concentration of monomeric Arginase was then mixed with dodecyl maltoside (DDM) to the critical micelle concentration (CMC) to reduce particle aggregation and used immediately afterwards to freeze grids. Then, 3 μL of each sample were applied to 1.2/1.3 grids (Au/Au Quantifoil or C/Cu C-flat), which have been previously plasma-cleaned using a Gatan Solarus (Pleasanton, California) and mounted in a Vitrobot Mark IV. The sample was then blotted with filter paper for six seconds and plunged in liquid ethane. Electron microscopy was performed using a ThermoFisher Titan Krios (Hillsboro, Oregon) transmission electron microscope operated at 300 kV and equipped with a Gatan Quantum 967 LS imaging filter and Gatan K2 Summit direct detector. Automated data-collection was carried out using Leginon software (Suloway et al., J. Struct. Biol. 151: 41-60 (2005)) in counting mode, collecting between 1500 and 3000 movies per sample at a defocus range between −1.0 and −2.0 μm, calibrated pixel size of 1.04 Å/pix and total dose of 45 e⁻/Å(Suloway et al., Ibid.).

Movies were aligned using MotionCor2 (Zheng et al., Nat. Methods 14: 331-332 (2017)) and the contrast transfer function (CTF) calculated using CTFFIND4 (Rohou. & Grigorieff, J. Struct. Biol. 192: 216-221 (2015)) within the Appion package (Lander et al., J. Struct. Biol. 166: 95-102 (2009)). Aligned micrographs were imported into cryoSPARC (Punjani et al., Nat. Methods 14: 290-296 (2017)) where all the subsequent steps of image processing were realized following a standard single particle workflow until the final reconstruction. Particle picking was performed based on blobs for mAb5. When the 2D classes from mAb5 became available, they were used as templates to select particles for the other samples. Several rounds of 2D classification ab-initio were performed to select the best-looking particles and separate the different oligomerization complexes when they existed. In all the cases non-uniform refinement and higher-order CTF refinement were used to generate the best reconstructions. Symmetries C3 or C1 were enforced during the final refinement when applicable. When used during the refinement, masks were generated in Chimera (Pettersen et al., J Comput Chem. 25: 1605-12 (2004)). Initial models for the structures were generated using MOE 2018.01 (Chemical Computing Group ULC, Montreal, QC, Canada) and the available structure of hArg (PDB ID=6V7C) (Mitcheltree. et al., ACS Med. Chem. Lett. 11, 582-588 (2020)). The structures were built in Coot (Emsley et al., Biol. Crystallog. 60: 2126-2132 (2004)) based on the cryoEM density maps and subjected to one round of real space refinement in Phenix (Afonine et al., Comput. Crystallogr. Newsl. 4: 43-44 (2013)). Summary of cryo-EM data collection is shown in Table 9 and statistics data processing, map generation, and model refinement values are shown Table 10. Table 11 summarizes the specific epitope-paratope interactions between hArg1 and mAb5.

TABLE 9
Summary of cryo-EM data
collection

| | |
|---|---|
| Sample | mAb5 |
| Microscope | Krios/EF |
| Camera | K2 |
| Pixel size (Å/pix) | 1.04 |
| Dose (e⁻/Å) | 45.44 |
| #images | 2048 |
| Defocus range | 1.0 to −2.0 |
| (μm) | |

TABLE 10
Statistics data processing,
map generation and model refinement

| | mAb5 |
|---|---|
| Sample | One trimer: three Fabs |
| #particles | 44345 |
| Global resolution (Å) | 3.1 |
| RMSD Bonds (Å) | 0.010 |
| RMSD Angles (Deg) | 1.08 |
| CC Mask | 0.83 |
| CC volume | 0.83 |
| CC peaks | 0.73 |
| Ramachandran | |
| Outliers %) | 0.74 |
| Allowed (%) | 12.4 |
| Favored (%) | 86.8 |
| All-atom clashscore | 7.2 |

TABLE 11
Amino acid interactions between the mAb5 paratope
and the hArg1 epitope.

| | hArg1 monomer A |
|---|---|
| Light Chain | |
| Ser30 | Pro54, Phe55 |
| Asn31 | Asp57 |
| Tyr32 | Phe55, Asp57 |
| Ser49 | Pro59 |
| Ala50 | Asp57, Pro59 |
| Thr53 | Pro59, Asn60 |
| Tyr91 | Asp57 |
| Heavy Chain | |
| Asp30, Asp31 | Arg21 |
| Asn52, Gly55 | Ser281 |
| Trp53 | Arg21, Gly22 |
| Asn54 | Ser281, Leu282 |
| Ser57 | Glu26 |
| Arg100 | Pro59 |
| Arg101 | Lys17, Asp57, Ile58, Pro59 |
| Arg102 | Pro20, Arg21, Lys68 |
| Gly 103 | Arg21, Gly22 |
| Tyr105 | Ser16, Lys17, Gln19, Pro20, Gly22, Glu25, Asn69 |
| Gly106 | Glu25 |

All interactions shown here are within 4 Å.
VH and VL amino acid position numbers are as set forth in SEQ ID NO: 2 and 3, respectively, and not according to Kabat.

### EXAMPLE 8

### Molecular Dynamics for mAb5

Initial Cartesian coordinates for the Arg1 and mAb5 systems were generated from the 3.1 Å resolution crystal structure. The system used for the simulation contained one monomer of Arg1 and the two mAb5 subunits (LC and the HC). The resulting structures were prepared using the leap module of the AMBER 16 package. The ternary system was solvated in a box of TIP3P (Jorgensen et al., J. Chem. Phys. (1983) doi: 10.1063/1.445869) water molecules extending at least 10 Å beyond the protein and chloride ions were added to maintain charge neutrality. The ff15ipq force field (Wang et al., J. Phys. Chem. B (2017) doi:10.1021/acs.jpcb.7b02320) was used to construct the topology files for the protein. The initial structure was conjugate gradient (CG) minimized for 200 steps for the water molecules only, followed by 10,000 steps of CG optimization of the entire system to remove any bad contacts. After minimization, the full system was gradually heated from 0 to 300 K using a constant NVT (number, volume, temperature) ensemble for 100 ps of MD (molecular dynamics) simulation. The system was then switched to a constant NPT (number, Pressure, temperature) ensemble at 300 K and one atm using a coupling value of 2.0 ps for both temperature and pressure and ran for 100 ps. Following equilibration, 100 ns of production data was collected at constant NPT using the graphics processing unit (GPU)-accelerated version of AMBER 16. Hydrogen bond analysis and distance calculations were computed using the CPPTRAJ program within AmberTools (Roe et al., J. Chem. Theory Comput. (2013) doi: 10.1021/ct400341p).

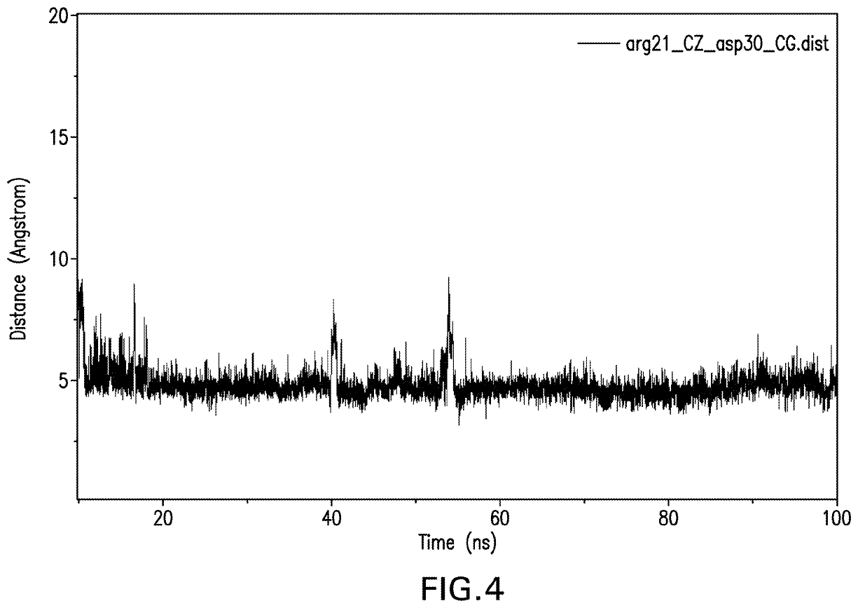

The results are shown in FIG. 4.

### EXAMPLE 9

### Ability of mAb5 to Form a 1:3 Complex

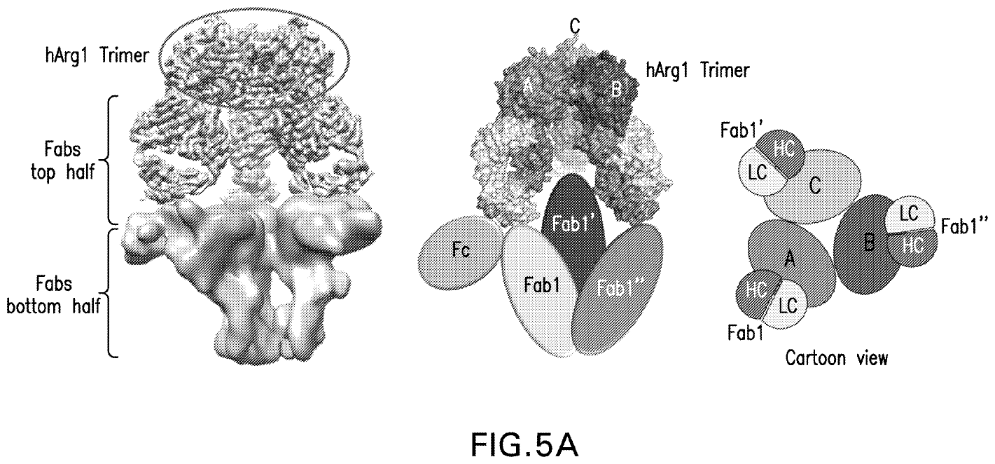

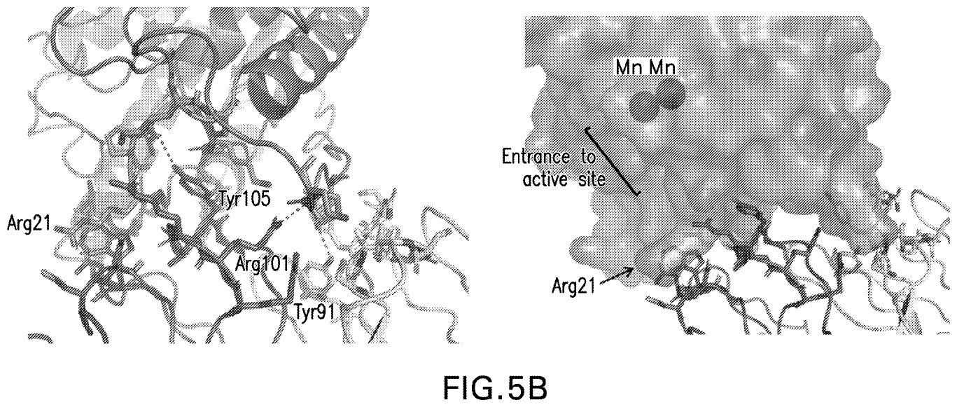

Using a transgenic mouse platform, mAb5 is a human antibody constructed on a mouse IgG1 D265A/kappa backbone. In this low-resolution portion of the map, the Fabs are oriented in a different conformation in which the termini of the antibodies are positioned significantly closer to each other. This suggests that no second hArg1 trimer is present and that the dominant form in the sample analyzed is one hArg1 trimer to three mAb5 antibodies, i.e. a 1:3 complex (FIG. 5A). It was observed that mAb5 binds to only a single monomer of the hArg1 trimer instead of spanning across two monomers (FIG. 5A) and therefore shares a relatively small interface with hArg1, covering only 908 Å² Table 8).

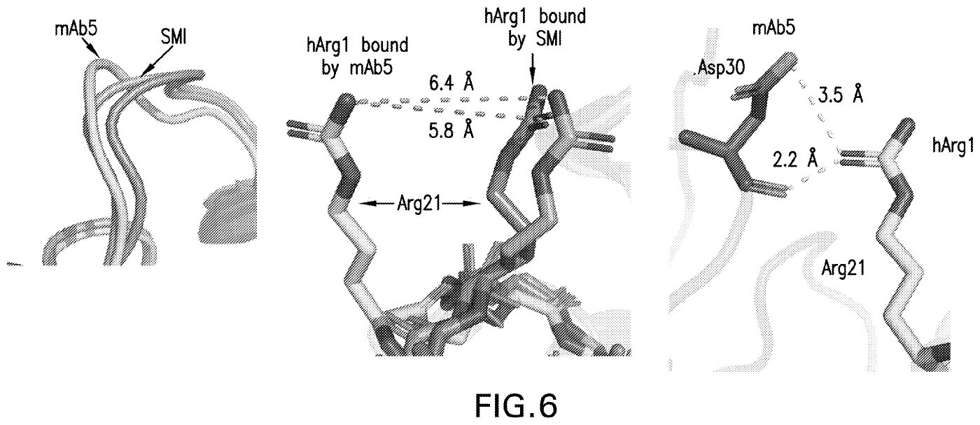

The electron density at the enzyme: antibody interface of the top half was very good and allowed for a complete assignment of the main chain and side chains of both hArg1 and mAb5. Analysis of the epitope: paratope interactions reveal that the binding of mAb5 does not sterically block the hArg1 active site, which remains accessible despite mAb5 being a neutralizing antibody. Additionally, when compared to previously determined small molecule-bound structures (Mitcheltree et al, ACS Med. Chem. Lett. 11, 582-588 (2020)) a small rearrangement of an hArg1 loop containing residues Lys16-Val24 is seen (FIG. 6, left panel). The inhibition of hArg1 occurs through a different mechanism of action as discussed below in which mAb5 interacts with an amino acid important to substrate binding by hArg1. Table 11 summarizes the residues involved in forming the epitope-paratope interactions.

Antibody mAb5 is a potent antibody in which the mechanism of inhibition is not obvious. The paratope is offset from the hArg1 active site and the active site remains accessible. The structure of mAb5 bound to hArg1 reveals a slight movement of an hArg1 loop containing residues Lys16-Val24 (FIG. 6, left and middle panels) as compared to small molecule (Mitcheltree et al., op.cit.) structures. Previous computational work on hArg1 has shown that when the active site opens in order to allow the entrance of substrate, residues Arg21 and Thr246, which sit on the outer lip of the active site entrance, are the main substrate interacting partners (Mortier et al., Sci. Rep. 7, 1-9 (2017)). It was also determined that product movement toward the hArg1 cavity exit is stabilized through an interaction with Arg21 (Mortier et al. Ibid). In the hArg1:mAb5 structure the loop containing Arg21 adopts an altered conformation in which residue Arg21 on hArg1 moves by approximately 6.1 Å (FIG. 6, middle panel). This is driven by Arg21 participating in a salt bridge with Asp30 of mAb5 (FIG. 6, right panel). A molecular dynamics simulation supports that Arg21 is strongly engaged by the antibody through a salt bridge with Asp30, suggesting it may not be available for binding to substrate and thereby inhibiting enzyme activity. The hydrogen bond between the guanidino donor of Arg21 and the carboxylate acceptor of Asp30 had an average distance of 2.8 Å and an angle of 153° .

The distance between the guanidino carbon atom (CZ of Arg21) and the carboxylate carbon (CG of Asp30) over the 100 ns simulation indicate the salt bridge is realized throughout the simulation time (FIG. 4). These results confirm that the ability of mAb5 to prevent the interaction of Arg21 with substrate is the likely mechanism of inhibition. This is a surface-based mechanism of competitive inhibition that could be used a template for the design of future inhibitors.

### EXAMPLE 10

### Comparison of Complex Sizes and Shapes

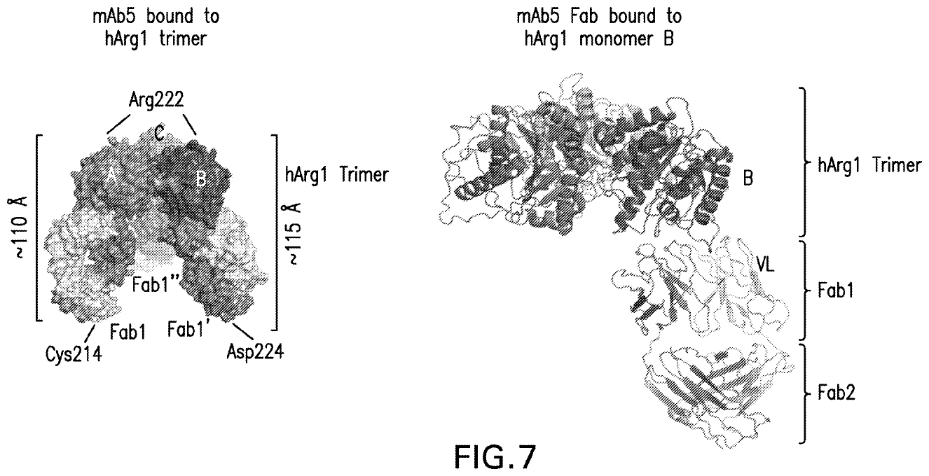

When considering the overall shape and size of the complexes (FIG. 7), it is clear that some of these differences are due to the varying backbone characteristics as described above. However, the epitope of the antibody is responsible for determining how and where the antibody binds to the hArg1 trimer which also plays a role in the antibody orientation and therefore the overall complex shape. Antibody mAb5 interacts with hArg1 mainly through its HC The length of the top half of the complex can be measured at about 110-115 Å including the hArg1 trimer, and the complex seems to take on a round appearance.

TABLE of
Sequences

| SEQ | | |
|---|---|---|
| ID | | |
| NO: | Description | Sequence |
| 1 | Human Arginase 1 | MSAKSRTIGIIGAPFSKGQPRGGVEEGPTVLRKAGLLEKLK |
| | | EQECDVKDYGDLPFADIPNDSPFQIVKNPRSVGKASEQLAG |
| | | KVAEVKKNGRISLVLGGDHSLAIGSISGHARVHPDLGVIWV |
| | | DAHTDINTPLTTTSGNLHGQPVSFLLKELKGKIPDVPGFSW |
| | | VTPCISAKDIVYIGLRDVDPGEHYILKTLGIKYFSMTEVDRL |
| | | GIGKVMEETLSYLLGRKKRPIHLSFDVDGLDPSFTPATGTP |
| | | VVGGLTYREGLYITEEIYKTGLLSGLDIMEVNPSLGKTPEEV |
| | | TRTVNTAVAITLACFGLAREGNHKPIDYLNPPK |
| 2 | VH | EVQLVESGGGVVRPGGSLRLSCAASGFTFDDYGMTWVRQ |
| | | APGKGLEWVSGINWNGGSTGYADSVKGRFTISRDNAKNSL |
| | | YLQMNSLRAEDTALYHCARDRRRGSYGSDAFDIWGQGTM |
| | | VTVSS |
| 3 | VL | DIQMTQSPSSLSASVGDRVTITCRASQGISNYLAWYQQKPG |
| | | KVPQLLISAASTLQSGVPSRFSGSGSGTDFTLTISSLQPEDVA |
| | | TYYCQKYNSAPRTFGQGTKVEIK |
| 4 | VH-CDR1 Kabat | DYGMT |
| 5 | VH-CDR2 | GINWNGGSTGYADSVKG |
| | Kabat/Kabat + | |
| | Chothia | |
| 6 | VH-CDR3 | DRRRGSYGSDAFDI |
| | Kabat/Chothia/ | |
| | Kabat + Chothia/AbM | |
| 7 | VH-CDR1 Chothia | GFTFDDY |
| 8 | VH-CDR2 Chothia | NWNGGS |
| 9 | VH-CDR1 | GFTFDDYGMT |
| | Kabat + Chothia/ | |
| | AbM | |
| 10 | VH-CDR2 AbM | GINWNGGSTG |
| 11 | VH-CDR1 IMGT | GFTFDDYG |
| 12 | VH-CDR2 IMGT | RRGSYGSDAFDI |
| 13 | VH-CDR3 IMGT | ARDRRRGSYGSDAFDI |
| 14 | VH-CDR1 Contact | DDYGMT |
| 15 | VH-CDR2 Contact | WVSGINWNGGSTG |
| 16 | VH-CDR3 Contact | ARDRRRGSYGSDAFD |
| 17 | VL-CDR1 | RASQGISNYLA |
| | Kabat/Chothia/ | |
| | Kabat + Chothia/AbM | |
| 18 | VL-CDR2 | AASTLQS |
| | Kabat/Chothia/ | |
| | Kabat + Chothia/AbM | |
| 19 | VL-CDR3 | QKYNSAPRT |
| | Kabat/Chothia/ | |
| | Kabat + Chothia/AbM/ | |
| | IMGT | |
| 20 | VL-CDR1 IMGT | QGISNY |
| 21 | VL-CDR1 Contact | SNYLAWY |
| 22 | VL-CDR2 Contact | LLISAASTLQ |
| 23 | VL-CDR3 Contact | QKYNSAPR |
| 24 | Human IgG1 HC | ASTKGPSVFPLAPSSKSTSGGTAALGCLVKDYFPEPVTVSW |
| | constant domain | NSGALTSGVHTFPAVLQSSGLYSLSSVVTVPSSSLGTQTYIC |
| | | NVNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 25 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (L234A L235A | NVNHKPSNTKVDKKVEPKSCDKTHTCPPCPAPEAAGGPSV |
| | D265S) | FLFPPKPKDTLMISRTPEVTCVVVSVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 26 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (L234A L235A | NVNHKPSNTKVDKKVEPKSCDKTHTCPPCPAPEAAGGPSV |
| | P329G) | FLFPPKPKDTLMISRTPEVTCVVVSVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 27 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (L235E) | NVNHKPSNTKVDKKVEPKSCDKTHTCPPCPAPELEGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 28 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (D265A) | NVNHKPSNTKVDKKVEPKSCDKTHTCPPCPAPELLGGPSVF |
| | | LFPPKPKDTLMISRTPEVTCVVVAVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 29 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (D265A N297G) | NVNHKPSNTKVDKKVEPKSCDKTHTCPPCPAPELLGGPSVF |
| | | LFPPKPKDTLMISRTPEVTCVVVAVSHEDPEVKFNWYVDG |
| | | VEVHNAKTKPREEQYGSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 30 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (E233A/L235A) | NVNHKPSNTKVDKKVEPKSCDKTHTCPPCPAPALAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 31 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (N297X, wherein X | NVNHKPSNTKVDKKVEPKSCDKTHTCPPCPAPELLGGPSVF |
| | is any amino acid | LFPPKPKDTLMISRTPEVTCVVVDVSHEDPEVKFNWYVDG |
| | other than N) | VEVHNAKTKPREEQYXSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 32 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFNSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 33 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (D265S) | CNVDHKPSNTKVDKTVERKCCVECPPCPAPPVAGPSVFLFP |
| | | PKPKDTLMISRTPEVTCVVVSVSHEDPEVQFNWYVDGVEV |
| | | HNAKTKPREEQFNSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 34 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (P329G) | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFNSTFRVVSVLTVVHQDWLNGKEYKCKV |
| | | SNKGLGAPIEKTISKTKGQPREPQVYTLPPSREEMTKNQVS |
| | | LTCLVKGFYPSDISVEWESNGQPENNYKTTPPMLDSDGSFF |
| | | LYSKLTVDKSRWQQGNVFSCSVMHEALHNHYTQKSLSLSP |
| | | GK |
| 35 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (D265A) | CNVDHKPSNTKVDKTVERKCCVECPPCPAPPVAGPSVFLFP |
| | | PKPKDTLMISRTPEVTCVVVAVSHEDPEVQFNWYVDGVEV |
| | | HNAKTKPREEQFNSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 36 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (D265A N297G) | CNVDHKPSNTKVDKTVERKCCVECPPCPAPPVAGPSVFLFP |
| | | PKPKDTLMISRTPEVTCVVVAVSHEDPEVQFNWYVDGVEV |
| | | HNAKTKPREEQFGSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 37 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (N297X, wherein X | CNVDHKPSNTKVDKTVERKCCVECPPCPAPPVAGPSVFLFP |
| | is any amino acid | PKPKDTLMISRTPEVTCVVVDVSHEDPEVQFNWYVDGVEV |
| | other than N) | HNAKTKPREEQFXSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 38 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (V234A G237A | CNVDHKPSNTKVDKTVERKCCVECPPCPAPPAAASSVFLFP |
| | P238S H268A | PKPKDTLMISRTPEVTCVVVDVSAEDPEVQFNWYVDGVEV |
| | V309L A330S | HNAKTKPREEQFNSTFRVVSVLTVLHQDWLNGKEYKCKV |
| | P331S | SNKGLPSSIEKTISKTKGQPREPQVYTLPPSREEMTKNQVSL |
| | X378S/A)(See | TCLVKGFYPSDIXVEWESNGQPENNYKTTPPMLDSDGSFFL |
| | IgGsigma SEQ ID | YSKLTVDKSRWQQGNVFSCSVMHEALHNHYTQKSLSLSP |
| | No: 78 in | GK |
| | WO2017079112) | |
| 39 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P) | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 40 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P P329G) | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLGSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LGK |
| 41 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P D265A) | CNVDHKPSNTKVDKRVESKYGPPCPPCPAPEFLGGPSVFLF |
| | | PPKPKDTLMISRTPEVTCVVVAVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 42 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P D265A | CNVDHKPSNTKVDKRVESKYGPPCPPCPAPEFLGGPSVFLF |
| | N297G) | PPKPKDTLMISRTPEVTCVVVAVSQEDPEVQFNWYVDGVE |
| | | VHNAKTKPREEQFGSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 43 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P N297X, | CNVDHKPSNTKVDKRVESKYGPPCPPCPAPEFLGGPSVFLF |
| | wherein X is any | PPKPKDTLMISRTPEVTCVVVDVSQEDPEVQFNWYVDGVE |
| | amino acid other | VHNAKTKPREEQFXSTYRVVSVLTVLHQDWLNGKEYKCK |
| | than N) | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 44 | Human LC Kappa | RTVAAPSVFIFPPSDEQLKSGTASVVCLLNNFYPREAKVQW |
| | Constant domain | KVDNALQSGNSQESVTEQDSKDSTYSLSSTLTLSKADYEK |
| | | HKVYACEVTHQGLSSPVTKSFNRGEC |
| 45 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (N297A/D356E/L358M) | NVNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYASTYRVVSVLTVLHQDWLNGKEYK |
| | | CKVSNKALPAPIEKTISKAKGQPREPQVYTLPPSREEMTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 46 | Human IgG1 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (L234F/L235E/P331S/ | CNVDHKPSNTKVDKRVESKYGPPCPPCPAPEFLGGPSVFLF |
| | D356E/L358M) | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSLTCLVKGFYP |
| | | SDIAVEWESNGQPENNYKTTPPVLDSDGSFFLYSRLTVDKS |
| | | RWQEGNVFSCSVMHEALHNHYTQKSLSLSLGK |
| 47 | Human LC lambda | GQPKANPTVTLFPPSSEELQANKATLVCLISDFYPGAVTVA |
| | Constant domain | WKADGSPVKAGVETTKPSKQSNNKYAASSYLSLTPEQWK |
| | | SHRSYSCQVTHEGSTVEKTVAPTECS |
| 48 | Mouse HC IgG1 | AKTTPPSVYPLAPGSAAQTNSMVTLGCLVKGYFPEPVTVT |
| | (D265A) constant | WNSGSLSSGVHTFPAVLQSDLYTLSSSVTVPSSTWPSETVT |
| | domain | CNVAHPASSTKVDKKIVPRDCGCKPCICTVPEVSSVFIFPPK |
| | | PKDVLTITLTPKVTCVVVAISKDDPEVQFSWFVDDVEVHTA |
| | | QTQPREEQFNSTFRSVSELPIMHQDWLNGKEFKCRVNSAAF |
| | | PAPIEKTISKTKGRPKAPQVYTIPPPKEQMAKDKVSLTCMIT |
| | | DFFPEDITVEWQWNGQPAENYKNTQPIMDTDGSYFVYSKL |
| | | NVQKSNWEAGNTFTCSVLHEGLHNHHTEKSLSHSPGK |
| 49 | Mouse HC IgG2 | AKTTAPSVYPLAPVCGDTTGSSVTLGCLVKGYFPEPVTLT |
| | constant domain | WNSGSLSSGVHTFPAVLQSDLYTLSSSVTVTSSTWPSQSITC |
| | | NVAHPASSTKVDKKIEPRGPTIKPCPPCKCPAPNLLGGPSVF |
| | | IFPPKIKDVLMISLSPIVTCVVVDVSEDDPDVQISWFVNNVE |
| | | VHTAQTQTHREDYNSTLRVVSALPIQHQDWMSGKEFKCK |
| | | VNNKDLPAPIERTISKPKGSVRAPQVYVLPPPEEEMTKKQV |
| | | TLTCMVTDFMPEDIYVEWTNNGKTELNYKNTEPVLDSDGS |
| | | YFMYSKLRVEKKNWVERNSYSCSVVHEGLHNHHTTKSFS |
| | | RTPGK |
| 50 | Mouse LC kappa | RTVAAPTVSIFPPSSEQLTSGGASVVCFLNNFYPKDINVKW |
| | constant domain | KIDGSERQNGVLNSWTDQDSKDSTYSMSSTLTLTKDEYER |
| | | HNSYTCEATHKTSTSPIVKSFNRNEC |
| 51 | Nucleotide sequence | GAGGTGCAGCTGGTGGAGAGCGGCGGCGGCGTGGTGAG |
| | encoding VH | ACCCGGCGGCAGCCTGAGACTGAGCTGCGCCGCCAGCG |
| | | GCTTCACCTTCGACGACTACGGCATGACCTGGGTGAGAC |
| | | AGGCCCCCGGCAAGGGCCTGGAGTGGGTGAGCGGCATC |
| | | AACTGGAACGGCGGCAGCACCGGCTACGCCGACAGCGT |
| | | GAAGGGCAGATTCACCATCAGCAGAGACAACGCCAAGA |
| | | ACAGCCTGTACCTGCAGATGAACAGCCTGAGAGCCGAG |
| | | GACACCGCCCTGTACCACTGCGCCAGAGACAGAAGAAG |
| | | AGGCAGCTACGGCAGCGACGCCTTCGACATCTGGGGCC |
| | | AGGGCACCATGGTGACCGTGAGCAGC |
| 52 | Nucleotide sequence | GACATCCAGATGACCCAGAGCCCCAGCAGCCTGAGCGC |
| | encoding VL | CAGCGTGGGCGACAGAGTGACCATCACCTGCAGAGCCA |
| | | GCCAGGGCATCAGCAACTACCTGGCCTGGTACCAGCAG |
| | | AAGCCCGGCAAGGTGCCCCAGCTGCTGATCAGCGCCGCC |
| | | AGCACCCTGCAGAGCGGCGTGCCCAGCAGATTCAGCGG |
| | | CAGCGGCAGCGGCACCGACTTCACCCTGACCATCAGCAG |
| | | CCTGCAGCCCGAGGACGTGGCCACCTACTACTGCCAGAA |
| | | GTACAACAGCGCCCCCAGAACCTTCGGCCAGGGCACCA |
| | | AGGTGGAGATCAAG |
| 53 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgGI HC constant | CTGCCTGGTGAAGGACTACTTCCCCGAGCCCGTGACCGT |
| | domain | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 54 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (L234A | GAGCTGGAACAGCGGCGCCCTGACCAGCGGCGTGCACA |
| | L235A D265S) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GGCCGCCGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGAGCGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 55 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (L234A | GAGCTGGAACAGCGGCGCCCTGACCAGCGGCGTGCACA |
| | L235A P329G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GGCCGCCGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGAGCGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGGGCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 56 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (L235E) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGGAGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 57 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (D265A) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGCCGTGAGCCACGAGGACCCCG |
| | | AGGTGAAGTTCAACTGGTACGTGGACGGCGTGGAGGTG |
| | | CACAACGCCAAGACCAAGCCCAGAGAGGAGCAGTACAA |
| | | CAGCACCTACAGAGTGGTGAGCGTGCTGACCGTGCTGCA |
| | | CCAGGACTGGCTGAACGGCAAGGAGTACAAGTGCAAGG |
| | | TGAGCAACAAGGCCCTGCCCGCCCCCATCGAGAAGACC |
| | | ATCAGCAAGGCCAAGGGCCAGCCCAGAGAGCCCCAGGT |
| | | GTACACCCTGCCCCCCAGCAGAGACGAGCTGACCAAGA |
| | | ACCAGGTGAGCCTGACCTGCCTGGTGAAGGGCTTCTACC |
| | | CCAGCGACATCGCCGTGGAGTGGGAGAGCAACGGCCAG |
| | | CCCGAGAACAACTACAAGACCACCCCCCCCGTGCTGGAC |
| | | AGCGACGGCAGCTTCTTCCTGTACAGCAAGCTGACCGTG |
| | | GACAAGAGCAGATGGCAGCAGGGCAACGTGTTCAGCTG |
| | | CAGCGTGATGCACGAGGCCCTGCACAACCACTACACCCA |
| | | GAAGAGCCTGAGCCTGAGCCCCGGCAAG |
| 58 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (D265A | GAGCTGGAACAGCGGCGCCCTGACCAGCGGCGTGCACA |
| | N297G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGCCGTGAGCCACGAGGACCCCG |
| | | AGGTGAAGTTCAACTGGTACGTGGACGGCGTGGAGGTG |
| | | CACAACGCCAAGACCAAGCCCAGAGAGGAGCAGTACGG |
| | | CAGCACCTACAGAGTGGTGAGCGTGCTGACCGTGCTGCA |
| | | CCAGGACTGGCTGAACGGCAAGGAGTACAAGTGCAAGG |
| | | TGAGCAACAAGGCCCTGCCCGCCCCCATCGAGAAGACC |
| | | ATCAGCAAGGCCAAGGGCCAGCCCAGAGAGCCCCAGGT |
| | | GTACACCCTGCCCCCCAGCAGAGACGAGCTGACCAAGA |
| | | ACCAGGTGAGCCTGACCTGCCTGGTGAAGGGCTTCTACC |
| | | CCAGCGACATCGCCGTGGAGTGGGAGAGCAACGGCCAG |
| | | CCCGAGAACAACTACAAGACCACCCCCCCCGTGCTGGAC |
| | | AGCGACGGCAGCTTCTTCCTGTACAGCAAGCTGACCGTG |
| | | GACAAGAGCAGATGGCAGCAGGGCAACGTGTTCAGCTG |
| | | CAGCGTGATGCACGAGGCCCTGCACAACCACTACACCCA |
| | | GAAGAGCCTGAGCCTGAGCCCCGGCAAG |
| 59 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain | GAGCTGGAACAGCGGCGCCCTGACCAGCGGCGTGCACA |
| | (E233A/L235A) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGC |
| | | CCTGGCCGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 60 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (N297X, | GAGCTGGAACAGCGGCGCCCTGACCAGCGGCGTGCACA |
| | wherein X is any | CCTTCCCCGCCGTGCTGCAGAGCAGCGGCCTGTACAGCC |
| | amino acid other | TGAGCAGCGTGGTGACCGTGCCCAGCAGCAGCCTGGGC |
| | than N) | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACN |
| | | NNAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 61 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGACGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 62 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (D265S) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGAGCGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 63 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (P329G) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGACGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGGGCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 64 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (D265A) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGCCGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 65 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (D265A | GAGCTGGAACAGCGGCGCCCTGACCAGCGGCGTGCACA |
| | N297G) | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGCCGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCGGCAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 66 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (N297X, | GAGCTGGAACAGCGGCGCCCTGACCAGCGGCGTGCACA |
| | wherein X is any | CCTTCCCCGCCGTGCTGCAGAGCAGCGGCCTGTACAGCC |
| | amino acid other | TGAGCAGCGTGGTGACCGTGCCCAGCAGCAACTTCGGCA |
| | than N) | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGACGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCNNNAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 67 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (V234A | GAGCTGGAACAGCGGCGCCCTGACCAGCGGCGTGCACA |
| | G237A P238S | CCTTCCCCGCCGTGCTGCAGAGCAGCGGCCTGTACAGCC |
| | H268A V309L | TGAGCAGCGTGGTGACCGTGCCCAGCAGCAACTTCGGCA |
| | A330S P331S | CCCAGACCTACACCTGCAACGTGGACCACAAGCCCAGC |
| | X378S/A)(See | AACACCAAGGTGGACAAGACCGTGGAGAGAAAGTGCTG |
| | IgGsigma SEQ ID | CGTGGAGTGCCCCCCCTGCCCCGCCCCCCCCGCCGCCGC |
| | No: 78 in | CAGCAGCGTGTTCCTGTTCCCCCCCAAGCCCAAGGACAC |
| | WO2017079112) | CCTGATGATCAGCAGAACCCCCGAGGTGACCTGCGTGGT |
| | | GGTGGACGTGAGCGCCGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGCTGCACCAGGACTGGCT |
| | | GAACGGCAAGGAGTACAAGTGCAAGGTGAGCAACAAGG |
| | | GCCTGCCCAGCAGCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCN |
| | | NNGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 68 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 69 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | P329G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGGGCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| 70 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | D265A) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGCCGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 71 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | D265A N297G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGCCGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCGGCAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 72 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | N297X, wherein X | CCTTCCCCGCCGTGCTGCAGAGCAGCGGCCTGTACAGCC |
| | is any amino acid | TGAGCAGCGTGGTGACCGTGCCCAGCAGCAGCCTGGGC |
| | other than N) | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCNNNAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 73 | Nucleotide sequence | AGAACCGTGGCCGCCCCCAGCGTGTTCATCTTCCCCCCC |
| | encoding human LC | AGCGACGAGCAGCTGAAGAGCGGCACCGCCAGCGTGGT |
| | Kappa Constant | GTGCCTGCTGAACAACTTCTACCCCAGAGAGGCCAAGGT |
| | domain | GCAGTGGAAGGTGGACAACGCCCTGCAGAGCGGCAACA |
| | | GCCAGGAGAGCGTGACCGAGCAGGACAGCAAGGACAGC |
| | | ACCTACAGCCTGAGCAGCACCCTGACCCTGAGCAAGGCC |
| | | GACTACGAGAAGCACAAGGTGTACGCCTGCGAGGTGAC |
| | | CCACCAGGGCCTGAGCAGCCCCGTGACCAAGAGCTTCA |
| | | ACAGAGGCGAGTGC |
| 74 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain | GAGCTGGAACAGCGGCGCCCTGACCAGCGGCGTGCACA |
| | (N297A/D356E/L358M) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACG |
| | | CCAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGAGGAGATGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 75 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG1 HC Constant | CTGCCTGGTGAAGGACTACTTCCCCGAGCCCGTGACCGT |
| | domain | GAGCTGGAACAGCGGCGCCCTGACCAGCGGCGTGCACA |
| | (L234F/L235E/P331S/ | CCTTCCCCGCCGTGCTGCAGAGCAGCGGCCTGTACAGCC |
| | D356E/L358M) | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCTGACCTGCCTGGTGAAGGGCTTCTACCC |
| | | CAGCGACATCGCCGTGGAGTGGGAGAGCAACGGCCAGC |
| | | CCGAGAACAACTACAAGACCACCCCCCCCGTGCTGGAC |
| | | AGCGACGGCAGCTTCTTCCTGTACAGCAGACTGACCGTG |
| | | GACAAGAGCAGATGGCAGGAGGGCAACGTGTTCAGCTG |
| | | CAGCGTGATGCACGAGGCCCTGCACAACCACTACACCCA |
| | | GAAGAGCCTGAGCCTGAGCCTGGGCAAG |
| 76 | Nucleotide sequence | GGCCAGCCCAAGGCCAACCCCACCGTGACCCTGTTCCCC |
| | encoding human LC | CCCAGCAGCGAGGAGCTGCAGGCCAACAAGGCCACCCT |
| | lambda Constant | GGTGTGCCTGATCAGCGACTTCTACCCCGGCGCCGTGAC |
| | domain | CGTGGCCTGGAAGGCCGACGGCAGCCCCGTGAAGGCCG |
| | | GCGTGGAGACCACCAAGCCCAGCAAGCAGAGCAACAAC |
| | | AAGTACGCCGCCAGCAGCTACCTGAGCCTGACCCCCGAG |
| | | CAGTGGAAGAGCCACAGAAGCTACAGCTGCCAGGTGAC |
| | | CCACGAGGGCAGCACCGTGGAGAAGACCGTGGCCCCCA |
| | | CCGAGTGCAGC |
| 77 | Leader peptide A | MSVPTQVLGLLLLWLTDARC |
| 78 | Leader peptide B | MEWSWVFLFFLSVTTGVHS |
| 79 | Nucleotide sequence | ATGAGCGTGCCCACCCAGGTGCTGGGCCTGCTGCTGCTG |
| | encoding leader | TGGCTGACCGACGCCAGATGC |
| | peptide A | |
| 80 | Nucleotide sequence | ATGGAGTGGAGCTGGGTGTTCCTGTTCTTCCTGAGCGTG |
| | encoding leader | ACCACCGGCGTGCACAGC |
| | peptide B | |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. An Arginase 1 binder comprising:
(a) three complementarity determining regions (CDRs) of an antibody heavy chain variable domain (VH) comprising the amino acid sequence set forth in SEQ ID NO: 2; and the three CDRs of an antibody light chain variable domain (VL) comprising the amino acid sequence set forth in SEQ ID NO: 3.

2. The Arginase 1 binder of claim 1, wherein the Arginase 1 binder specifically binds to an arginase 1 trimer to form a complex comprising three Arginase 1 binders and one arginase trimer, and inhibits arginase 1 activity.

3. The Arginase 1 binder of claim 1, wherein
(a) the VH CDRs comprise a VH-CDR1 comprising the amino acid sequence set forth in SEQ ID NO: 4, a VH-CDR2 comprising the amino acid sequence set forth in SEQ ID NO: 5, and a VH-CDR3 comprising the amino acid sequence set forth in SEQ ID NO: 6, and
(b) the VL CDRs comprise a VL-CDR1 comprising the amino acid sequence set forth in SEQ ID NO: 17, a VL-CDR2 comprising the amino acid sequence set forth in SEQ ID NO: 18, and a VL-CDR3 comprising the amino acid sequence set forth in SEQ ID NO: 19;
wherein the CDR sequences are defined by Kabat.

4. The Arginase 1 binder of claim 1, wherein the Arginase 1 binder comprises a VH comprising the amino acid sequence set forth in SEQ ID NO: 2 and a VL comprising the amino acid sequence set forth in SEQ ID NO: 3.

5. The Arginase 1 binder of claim 4, wherein the antibody further comprises a heavy chain constant domain of the IgG1, IgG2, IgG3, or IgG4 isotype.

6. The Arginase 1 binder of claim 5, wherein the heavy chain constant domain of the IgG1, IgG2, IgG3, or IgG4 isotype comprises an Fc domain comprising one or more mutations that render the Fc domain effector-silent.

7. An Arginase 1 binder that is an antibody or antigen binding fragment comprising two identical Fabs, a first Fab comprising a first heavy chain variable domain (VH) and a first light chain variable domain (VL) and a second Fab comprising a second VH and a second VL,
wherein the Arginase 1 binder binds to an arginase 1 trimer comprising three arginase 1 monomers,
wherein the first Fab VH and VL bind to an epitope of a monomer of the arginase 1 trimer and the second Fab VH and VL do not detectably bind an epitope of a monomer of an arginase 1 trimer.

8. The Arginase 1 binder of claim 7, wherein
(a) the VH of the first Fab binds to amino acids Arg21, Ser281, Gly22, Ser281, Leu282, Glu26, Pro59, Lys17, Asp57, Ile58, Pro59, Pro20, Lys68, Ser16,, Gln19, Glu25, Asn69, and Glu25 of the monomer of the arginase 1 trimer; and
(b) the VL of the first Fab binds to amino acids Pro54, Phe55, Asp57, Pro59, and Asn60 of the monomer of the of the first arginase 1 trimer.

9. The Arginase 1 binder of claim 8, wherein the Arginase 1 binder binds one arginase 1 trimer to form a complex comprising a three to one ratio of Arginase 1 binder to arginase 1 trimer.

10. A composition comprising an Arginase 1 binder of claims 1 and a pharmaceutically acceptable carrier.

11. A method for treating cancer or proliferative disease in an individual in need thereof comprising:
administering to the individual a therapeutically effective amount an Arginase 1 binder of claims 1 to treat the cancer or a proliferative disease.

12. An arginase 1 binder of claims 1 for treatment of cancer or proliferative disease.

13. (canceled)

14. A combination therapy for treating cancer or proliferative disease comprising an arginase 1 binder of claims 1 and a therapeutic agent.

15. The combination therapy of claim 14, wherein the therapeutic agent is a chemotherapy agent or a therapeutic antibody.

16. The combination therapy of claim 15, wherein the antibody is an anti-PD1 or anti-PD-L1 antibody.

17. A nucleic acid molecule encoding the VH of the Arginase 1 binder of claim 1 and/or VL of the Arginase 1 binder of claim 1.

18. An expression vector comprising one or more of the nucleic acid molecules of claim 18.

19. A host cell comprising the expression vector of claim 18.

20. A method for producing an Arginase 1 binder comprising:
(a) providing the host cell of claim 19;
(b) cultivating the host cell in a medium under conditions suitable for expressing the Arginase 1 binder; and
(c) isolating the Arginase 1 binder from the medium.


SEQUENCE LISTING (81 sequences)

SEQ ID NO: 1 - protein, 322 aa, Homo Sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - protein, 123 aa, Artificial Sequence
  VH
EVQLVESGGGVVRPGGSLRLSCAASGFTFDDYGMTWVRQAPGKGLEWVSGINWNGGSTGYADSVKGRFTISRDNAKNSLYLQMNSLRAEDTALYHCARDRRRGSYGSDAFDIWGQGTMVTVSS

SEQ ID NO: 3 - protein, 107 aa, Artificial Sequence
  VL
DIQMTQSPSSLSASVGDRVTITCRASQGISNYLAWYQQKPGKVPQLLISAASTLQSGVPSRFSGSGSGTDFTLTISSLQPEDVATYYCQKYNSAPRTFGQGTKVEIK

SEQ ID NO: 4 - protein, 5 aa, Artificial Sequence
  VH-CDR1 Kabat
DYGMT

SEQ ID NO: 5 - protein, 17 aa, Artificial Sequence
  VH-CDR2 Kabat/Kabat+Chothia
GINWNGGSTGYADSVKG

SEQ ID NO: 6 - protein, 14 aa, Artificial Sequence
  VH-CDR3 Kabat/Chothia/Kabat+Chothia/AbM
DRRRGSYGSDAFDI

SEQ ID NO: 7 - protein, 7 aa, Artificial Sequence
  VH-CDR1 Chothia
GFTFDDY

SEQ ID NO: 8 - protein, 6 aa, Artificial Sequence
  VH-CDR2 Chothia
NWNGGS

SEQ ID NO: 9 - protein, 10 aa, Artificial Sequence
  VH-CDR1 Kabat+Chothia/AbM
GFTFDDYGMT

SEQ ID NO: 10 - protein, 10 aa, Artificial Sequence
  VH-CDR2 AbM
GINWNGGSTG

SEQ ID NO: 11 - protein, 8 aa, Artificial Sequence
  VH-CDR1 IMGT
GFTFDDYG

SEQ ID NO: 12 - protein, 12 aa, Artificial Sequence
  VH-CDR2 IMGT
RRGSYGSDAFDI

SEQ ID NO: 13 - protein, 16 aa, Artificial Sequence
  VH-CDR3 IMGT
ARDRRRGSYGSDAFDI

SEQ ID NO: 14 - protein, 6 aa, Artificial Sequence
  VH-CDR1 Contact
DDYGMT

SEQ ID NO: 15 - protein, 13 aa, Artificial Sequence
  VH-CDR2 Contact
WVSGINWNGGSTG

SEQ ID NO: 16 - protein, 15 aa, Artificial Sequence
  VH-CDR3 Contact
ARDRRRGSYGSDAFD

SEQ ID NO: 17 - protein, 11 aa, Artificial Sequence
  VL-CDR1 Kabat/Chothia/Kabat+Chothia/AbM
RASQGISNYLA

SEQ ID NO: 18 - protein, 7 aa, Artificial Sequence
  VL-CDR2 Kabat/Chothia/Kabat+Chothia/AbM
AASTLQS

SEQ ID NO: 19 - protein, 9 aa, Artificial Sequence
  VL-CDR3 Kabat/Chothia/Kabat+Chothia/AbM/IMGT
QKYNSAPRT

SEQ ID NO: 20 - protein, 6 aa, Artificial Sequence
  VL-CDR1 IMGT
QGISNY

SEQ ID NO: 21 - protein, 7 aa, Artificial Sequence
  VL-CDR1 Contact
SNYLAWY

SEQ ID NO: 22 - protein, 10 aa, Artificial Sequence
  VL-CDR2 Contact
LLISAASTLQ

SEQ ID NO: 23 - protein, 8 aa, Artificial Sequence
  VL-CDR3 Contact
QKYNSAPR

SEQ ID NO: 24 - protein, 330 aa, Artificial Sequence [sequence omitted]

SEQ ID NO: 25 - protein, 330 aa, Artificial Sequence
  Human IgG1 HC Constant domain (L234A L235A D265S)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 330 aa, Artificial Sequence
  Human IgG1 HC Constant domain (L234A L235A P329G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 330 aa, Artificial Sequence
  Human IgG1 HC Constant domain (L235E)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 330 aa, Artificial Sequence
  Human IgG1 HC Constant domain (D265A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, Artificial Sequence
  Human IgG1 HC Constant domain (D265A N297G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, Artificial Sequence
  Human IgG1 HC Constant domain (E233A/L235A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, Artificial Sequence
  Human IgG1 HC Constant domain (N297X
  MISC_FEATURE (180)..(180): Xaa is any amino acid other than Asn
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 326 aa, Artificial Sequence
  Human IgG2 HC Constant domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 33 - protein, 326 aa, Artificial Sequence
  Human IgG2 HC Constant domain (D265S)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVSVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 34 - protein, 326 aa, Artificial Sequence
  Human IgG2 HC Constant domain (P329G)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLGAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 35 - protein, 326 aa, Artificial Sequence
  Human IgG2 HC Constant domain (D265A)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 36 - protein, 326 aa, Artificial Sequence
  Human IgG2 HC Constant domain (D265A N297G)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFGSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 37 - protein, 326 aa, Artificial Sequence
  Human IgG2 HC Constant domain (N297X)
  MISC_FEATURE (176)..(176): Xaa is any amino acid other than Asn
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFXSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 38 - protein, 326 aa, Artificial Sequence
  Human IgG2 HC Constant domain (V234A G237A P238S H268A V309L A330S P331S X378S/A)
  misc_feature (257)..(257): Xaa can be any naturally occurring amino acid
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIXVEWESNGQPENNYKTTPPMLDSDGSFFLYSKLTVDKSRWQQGNVFSCSVMHEALHNHYTQKSLSLSPGK

SEQ ID NO: 39 - protein, 327 aa, Artificial Sequence
  Human IgG4 HC Constant domain (S228P)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 327 aa, Artificial Sequence
  Human IgG4 HC Constant domain (S228P P329G)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 327 aa, Artificial Sequence
  Human IgG4 HC Constant domain (S228P D265A)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 327 aa, Artificial Sequence
  Human IgG4 HC Constant domain (S228P D265A N297G)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 327 aa, Artificial Sequence
  Human IgG4 HC Constant domain (S228P N297X)
  MISC_FEATURE (177)..(177): Xaa is any amino acid other than Asn
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 107 aa, Artificial Sequence [sequence omitted]

SEQ ID NO: 45 - protein, 330 aa, Artificial Sequence
  Human IgG1 HC Constant domain (N297A/D356E/ L358M)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 317 aa, Artificial Sequence
  Human IgG1 HC Constant domain (L234F/L235E/ P331S/D356E/L358M)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 106 aa, Artificial Sequence
  Human LC lambda Constant domain
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 48 - protein, 324 aa, Artificial Sequence
  Mouse HC IgG1 (D265A) constant domain
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 49 - protein, 330 aa, Artificial Sequence
  Mouse HC IgG2 constant domain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 50 - protein, 107 aa, Artificial Sequence
  Mouse LC kappa constant domain
RTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 51 - DNA, 375 bp, Artificial Sequence
  Nucleotide sequence encoding VH1
caggtgcagctggtgcagagcggcgccgaggtgaagaagcccggcgccagcgtgaaggtgagctgcaaggccagcggctacaccttcaccaactacggcatcagctgggtgagacaggcccccggccagggcctggagtggatgggctggatcagcgcctacaacggcaacaccaactacgcccagaagctgcagggcagagtgaccatgaccaccgacaccagcaccagcaccgcctacatggagctgagaagcctgagaagcgacgacaccgccgtgtactactgcgccagagagggcgcctacggctacagaagcccctaccacaactggttcgacccctggggccagggcaccctggtgaccgtgagcagc

SEQ ID NO: 52 - DNA, 375 bp, Artificial Sequence
  Nucleotide sequence encoding VH2
gaggtgcagctggtgcagagcggcgccgaggtgaagaagcccggcgccagcgtgaaggtgagctgcaaggccagcggctacaccttcttcaagtacggcatcagctgggtgagacaggcccccggccagggcctggagtggatgggcagcatcagcccctacaccggcgagacccactacgcccagaagctgcagggcagagtgaccatgaccaccgacaccagcaccagcaccgcctacatggagctgagaagcctgagaagcgacgacaccgccgtgtactactgcgccagagagggcgcctacggctacagaagcccctaccagaactggttcgacccctggggccagggcaccctggtgaccgtgagcagc

SEQ ID NO: 53 - DNA, 321 bp, Artificial Sequence
  Nucleotide sequence encoding VL
gagatcgtgatgacccagagccccgccaccctgagcctgagccccggcgagagagccaccctgagctgcagagccagccagagcgtgagcagctacctggcctggtaccagcagaagcccggccaggcccccagactgctgatctacgacgccagcaacagagccaccggcatccccgccagattcagcggcagcggcagcggcaccgacttcaccctgaccatcagcagcctggagcccgaggacttcgccgtgtactactgccagcagcacagcctgctgcccagaaccttcggcggcggcaccaaggtggagatcaag

SEQ ID NO: 54 - DNA, 990 bp, Artificial Sequence
  Nucleotide sequence encoding human IgG1 HC constant domain
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 55 - DNA, 990 bp, Artificial Sequence
  Nucleotide sequence encoding human IgG1 HC Constant domain (L234A L235A D265S)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgaggccgccggcggccccagcgtgttcctgttcccccccaagcccaaggacaccctgatgatcagcagaacccccgaggtgacctgcgtggtggtgagcgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 56 - DNA, 990 bp, Artificial Sequence
  Nucleotide sequence encoding human IgG1 HC Constant domain (L234A L235A P329G)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgaggccgccggcggccccagcgtgttcctgttcccccccaagcccaaggacaccctgatgatcagcagaacccccgaggtgacctgcgtggtggtgagcgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgggcgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 57 - DNA, 990 bp, Artificial Sequence
  Nucleotide sequence encoding human IgG1 HC Constant domain (L235E)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctggagggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 58 - DNA, 990 bp, Artificial Sequence
  Nucleotide sequence encoding human IgG1 HC Constant domain (D265A)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggccgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 59 - DNA, 990 bp, Artificial Sequence
  Nucleotide sequence encoding human IgG1 HC Constant domain (D265A N297G)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggccgtgagccacgaggaccccgaggtgaagttcaactggtacgtggacggcgtggaggtgcacaacgccaagaccaagcccagagaggagcagtacggcagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 60 - DNA, 990 bp, Artificial Sequence
  Nucleotide sequence encoding human IgG1 HC Constant domain (E233A/L235A)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgccctggccggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 61 - DNA, 990 bp, Artificial Sequence
  Nucleotide sequence encoding human IgG1 HC Constant domain (N297X, wherein X is any amino acid other than N)
  misc_feature (538)..(540): Codon wherein each N is any nucleotide with the proviso that the codon does not encode Asn or form a translation stop codon
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacnnnagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 62 - DNA, 978 bp, Artificial Sequence
  Nucleotide sequence encoding human IgG2 HC Constant domain
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggacgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 63 - DNA, 978 bp, Artificial Sequence
  Nucleotide sequence encoding human IgG2 HC Constant domain (D265S)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtgagcgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 64 - DNA, 978 bp, Artificial Sequence
  Nucleotide sequence encoding human IgG2 HC Constant domain (P329G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggacgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgggcgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 65 - DNA, 978 bp, Artificial Sequence
  Nucleotide sequence encoding human IgG2 HC Constant domain (D265A)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggccgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 66 - DNA, 978 bp, Artificial Sequence
  Nucleotide sequence encoding human IgG2 HC Constant domain (D265A N297G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggccgtgagccacgaggaccccgaggtgcagttcaactggtacgtggacggcgtggaggtgcacaacgccaagaccaagcccagagaggagcagttcggcagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 67 - DNA, 978 bp, Artificial Sequence
  Nucleotide sequence encoding human IgG2 HC Constant domain (N297X, wherein X is any amino acid other than N)
  misc_feature (526)..(528): Codon wherein each N is any nucleotide with the proviso that the codon does not encode Asn or form a translation stop codon
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggacgtgagccacgaggaccccgaggtgcagttcaactggtacgtggacggcgtggaggtgcacaacgccaagaccaagcccagagaggagcagttcnnnagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 68 - DNA, 978 bp, Artificial Sequence
  Nucleotide sequence encoding human IgG2 HC Constant domain (V234A G237A P238S H268A V309L A330S P331S X378S/ A)
  misc_feature (769)..(771): n is a, c, g, or t
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgccgccgccagcagcgtgttcctgttcccccccaagcccaaggacaccctgatgatcagcagaacccccgaggtgacctgcgtggtggtggacgtgagcgccgaggaccccgaggtgcagttcaactggtacgtggacggcgtggaggtgcacaacgccaagaccaagcccagagaggagcagttcaacagcaccttcagagtggtgagcgtgctgaccgtgctgcaccaggactggctgaacggcaaggagtacaagtgcaaggtgagcaacaagggcctgcccagcagcatcgagaagaccatcagcaagaccaagggccagcccagagagccccaggtgtacaccctgccccccagcagagaggagatgaccaagaaccaggtgagcctgacctgcctggtgaagggcttctaccccagcgacatcnnngtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 69 - DNA, 981 bp, Artificial Sequence
  Nucleotide sequence encoding human IgG4 HC Constant domain (S228P)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 70 - DNA, 960 bp, Artificial Sequence
  Nucleotide sequence encoding human IgG4 HC Constant domain (S228P P329G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgggcagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagc

SEQ ID NO: 71 - DNA, 981 bp, Artificial Sequence
  Nucleotide sequence encoding human IgG4 HC Constant domain (S228P D265A)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggccgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 72 - DNA, 981 bp, Artificial Sequence
  Nucleotide sequence encoding human IgG4 HC Constant domain (S228P D265A N297G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggccgtgagccaggaggaccccgaggtgcagttcaactggtacgtggacggcgtggaggtgcacaacgccaagaccaagcccagagaggagcagttcggcagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 73 - DNA, 981 bp, Artificial Sequence
  Nucleotide sequence encoding human IgG4 HC Constant domain (S228P N297X, wherein X is any amino acid other than N)
  misc_feature (529)..(531): Codon wherein each N is any nucleotide with the proviso that the codon does not encode Asn or form a transaltion stop codon
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcnnnagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 74 - DNA, 321 bp, Artificial Sequence
  Nucleotide sequence encoding human LC Kappa Constant domain
agaaccgtggccgcccccagcgtgttcatcttcccccccagcgacgagcagctgaagagcggcaccgccagcgtggtgtgcctgctgaacaacttctaccccagagaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacagaggcgagtgc

SEQ ID NO: 75 - DNA, 990 bp, Artificial Sequence
  Nucleotide sequence encoding human IgG1 HC Constant domain (N297A/D356E/L358M)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacgccagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 76 - DNA, 951 bp, Artificial Sequence
  Nucleotide sequence encoding human IgG1 HC Constant domain (L234F/L235E/P331S/D356E/L358M)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 77 - DNA, 318 bp, Artificial Sequence
  Nucleotide sequence encoding human LC lambda Constant domain
ggccagcccaaggccaaccccaccgtgaccctgttcccccccagcagcgaggagctgcaggccaacaaggccaccctggtgtgcctgatcagcgacttctaccccggcgccgtgaccgtggcctggaaggccgacggcagccccgtgaaggccggcgtggagaccaccaagcccagcaagcagagcaacaacaagtacgccgccagcagctacctgagcctgacccccgagcagtggaagagccacagaagctacagctgccaggtgacccacgagggcagcaccgtggagaagaccgtggcccccaccgagtgcagc

SEQ ID NO: 78 - protein, 20 aa, Artificial Sequence
  Leader peptide A
MSVPTQVLGLLLLWLTDARC

SEQ ID NO: 79 - protein, 19 aa, Artificial Sequence
  Leader peptide B
MEWSWVFLFFLSVTTGVHS

SEQ ID NO: 80 - DNA, 60 bp, Artificial Sequence
  Nucleotide sequence encoding leader peptide A
atgagcgtgcccacccaggtgctgggcctgctgctgctgtggctgaccgacgccagatgc

SEQ ID NO: 81 - DNA, 57 bp, Artificial Sequence
  Nucleotide sequence encoding leader peptide B
atggagtggagctgggtgttcctgttcttcctgagcgtgaccaccggcgtgcacagc